SEQUENCE LISTING (42 sequences)

SEQ ID NO: 1 - protein, 986 aa, synthetic construct
TRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFK

SEQ ID NO: 2 - protein, 198 aa, synthetic construct
EASPASGPRHLMDPHIFTSNFNNGIGRHKTYLCYEVERLDNGTSVKMDQHRGFLHGQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 3 - protein, 299 aa, synthetic construct
ETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDL

SEQ ID NO: 4 - protein, 83 aa, synthetic construct
TNLSDIIEKETGKQLVIQESILMLPEEVEEVIGNKPESDILVHTAYDESTDENVMLLTSDAPEYKPWALVIQDSNGENKIKML

SEQ ID NO: 5 - protein, 16 aa, synthetic construct
SGSETPGTSESATPES

SEQ ID NO: 6 - protein, 7 aa, synthetic construct
PKKKRKV

SEQ ID NO: 7 - protein, 9 aa, synthetic construct
PAAKRVKLD

SEQ ID NO: 8 - protein, 1780 aa, synthetic construct
MPKKKRKVDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKHMSGSETPGTSESATPESEASPASGPRHLMDPHIFTSNFNNGIGRHKTYLCYEVERLDNGTSVKMDQHRGFLHGQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNGSGSETPGTSESATPESPWETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDHPQIIKKGPGIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSPKKKRKVPGYPYDVPDYAGSAAPAAKRVKLDGGSGGGSGSGPAAKRVKLDGPRLEVLFQGPGLEHHHHHH

SEQ ID NO: 9 - protein, 1751 aa, synthetic construct
MPKKKRKVDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKHMGSEASPASGPRHLMDPHIFTSNFNNGIGRHKTYLCYEVERLDNGTSVKMDQHRGFLHGQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNGSPWETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDHPQIIKKGPGIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSPKKKRKVPGYPYDVPDYAGSAAPAAKRVKLDGGSGGGSGSGPAAKRVKLDGPRLEVLFQGPGLEHHHHHH

SEQ ID NO: 10 - protein, 9 aa, synthetic construct
PAAKKKKLD

SEQ ID NO: 11 - protein, 18 aa, synthetic construct
KRPAATKKAGQAKKKKLD

SEQ ID NO: 12 - protein, 20 aa, synthetic construct
KRTADGSEFESTPKKKRKVE

SEQ ID NO: 13 - protein, 23 aa, synthetic construct
PRRRPLHSSAMEVQTKKVRKVPP

SEQ ID NO: 14 - RNA, 82 nt, synthetic construct
  source 1..82: mol_type = other RNA
gttttagagctagaaatagcaagttaaaataaaggctagtccgttatcaacttgaaaaagtggcaccgagtcggtgcttttt

SEQ ID NO: 15 - RNA, 80 nt, synthetic construct
  source 1..80: mol_type = other RNA
gttttagagctagaaatagcaagttaaaataaggctagtccgttatcaacttgaaaaagtggcaccgagtcggtgctttt

SEQ ID NO: 16 - DNA, 20 bp, synthetic construct
  source 1..20: mol_type = other DNA
tttatcacaggctccaggaa

SEQ ID NO: 17 - DNA, 20 bp, synthetic construct
  source 1..20: mol_type = other DNA
ttgcttttatcacaggctcc

SEQ ID NO: 18 - DNA, 20 bp, synthetic construct
  source 1..20: mol_type = other DNA
agagagccttccgaaagagg

SEQ ID NO: 19 - DNA, 22 bp, synthetic construct
  source 1..22: mol_type = other DNA
gccagaaaagagatatggcatc

SEQ ID NO: 20 - protein, 32 aa, synthetic construct
SGGSSGGSSGSETPGTSESATPESSGGSSGGS

SEQ ID NO: 21 - DNA, 20 bp, unidentified
  source 1..20: mol_type = unassigned DNA
aagttcgctaatcccggaac

SEQ ID NO: 22 - DNA, 20 bp, unidentified
  source 1..20: mol_type = unassigned DNA
gaccctcagccgtgctgctc

SEQ ID NO: 23 - DNA, 20 bp, unidentified
  source 1..20: mol_type = unassigned DNA
gtcccctccaccccacagtg

SEQ ID NO: 24 - DNA, 23 bp, unidentified
  source 1..23: mol_type = unassigned DNA
ggaatcccttctgcagcacctgg

SEQ ID NO: 25 - DNA, 23 bp, unidentified
  source 1..23: mol_type = unassigned DNA
gaccccctccaccccgcctccgg

SEQ ID NO: 26 - DNA, 23 bp, unidentified
  source 1..23: mol_type = unassigned DNA
cgtcgccgatcttcacagggtgg

SEQ ID NO: 27 - DNA, 23 bp, unidentified
  source 1..23: mol_type = unassigned DNA
gagtccgagcagaagaagaaggg

SEQ ID NO: 28 - DNA, 23 bp, unidentified
  source 1..23: mol_type = unassigned DNA
aagttcgctaatcccggaactgg

SEQ ID NO: 29 - DNA, 23 bp, unidentified
  source 1..23: mol_type = unassigned DNA
gtcatcttagtcattacctgagg

SEQ ID NO: 30 - DNA, 23 bp, unidentified
  source 1..23: mol_type = unassigned DNA
gaccctcagccgtgctgctcggg

SEQ ID NO: 31 - DNA, 23 bp, unidentified
  source 1..23: mol_type = unassigned DNA
ggggctcaacatcggaagagggg

SEQ ID NO: 32 - DNA, 23 bp, unidentified
  source 1..23: mol_type = unassigned DNA
gtcccctccaccccacagtgggg

SEQ ID NO: 33 - DNA, 23 bp, unidentified
  source 1..23: mol_type = unassigned DNA
gagtctaagcagaagaagaagag

SEQ ID NO: 34 - DNA, 22 bp, unidentified
  source 1..22: mol_type = unassigned DNA
gagtccttgagaagaaggaagg

SEQ ID NO: 35 - DNA, 23 bp, unidentified
  source 1..23: mol_type = unassigned DNA
acgtctgagcagaagaagaatgg

SEQ ID NO: 36 - DNA, 23 bp, unidentified
  source 1..23: mol_type = unassigned DNA
gaccccccccaccccgcccccgg

SEQ ID NO: 37 - DNA, 23 bp, unidentified
  source 1..23: mol_type = unassigned DNA
tgccccccccaccccacctctgg

SEQ ID NO: 38 - DNA, 23 bp, unidentified
  source 1..23: mol_type = unassigned DNA
attcccccccaccccgcctcagg

SEQ ID NO: 39 - DNA, 20 bp, synthetic construct
  source 1..20: mol_type = other DNA
tttatctcaggctccaggaa

SEQ ID NO: 40 - DNA, 23 bp, unidentified
  source 1..23: mol_type = unassigned DNA
gaccctcagccagtgctgctcgg

SEQ ID NO: 41 - DNA, 23 bp, unidentified
  source 1..23: mol_type = unassigned DNA
tttatcacaggctccaggaaggg

SEQ ID NO: 42 - DNA, 23 bp, unidentified
  source 1..23: mol_type = unassigned DNA
ttgcttttatcacaggctccagg

(19) United States
(12) Patent Application Publication
(10) Pub. No.: US 2026/0308553 A1
(43) Pub. Date: Oct. 8, 2026

(54) Cas-embedded Cytidine Deaminase Ribonucleoprotein Complexes having Improved Base Editing Specificity and Efficiency

(71) Applicant: University of Massachusetts, Boston, MA (US)

(72) Inventors: Jeong Min Lee, Shrewsbury, MA (US); Nese Kurt Yilmaz, Shrewsbury, MA (US); Celia A. Schiffer, Shrewsbury, MA (US)

(21) Appl. No.: 19/478,952

(22) PCT Filed: Feb. 27, 2024
(86) PCT No.: PCT/US2024/017427

§ 371 Date: Oct. 27, 2025

Related U.S. Application Data
(60) Provisional application No. 63/498,760, filed on Apr. 27, 2023.

(51) Int. Cl. C12N 9/22; C07K 14/47; C12N 9/78

(52) U.S. Cl. CPC C12N 9/226; C07K 14/4703; C12N 9/78; C07K 2319/09; C07K 2319/80; C12Y 305/04005

(57) ABSTRACT

Some aspects of the disclosure provide Cas-embedded cytosine base editors with expanded or shifted editing windows, allowing for editing of cytosines (Cs) at otherwise inaccessible positions. Methods of treating a disease or disorder using the Cas-embedded cytosine base editors are also provided.

### CROSS-REFERENCE TO RELATED APPLICATIONS

The present application claims priority from U.S. provisional application Ser. No. 63/498,760, filed Apr. 27, 2023, the contents of which are hereby incorporated herein by reference in their entirety.

### GOVERNMENT RIGHTS IN INVENTION

This invention was made with government support under AI150478 awarded by the National Institutes of Health. The government has certain rights in the invention.

### TECHNICAL FIELD

The present invention relates to Cas-embedded cytidine deaminase fusion proteins and complexes thereof, and more particularly to Cas-embedded eA3A ribonucleoprotein complexes having improved base editing specificity and efficiency.

### BACKGROUND ART

Cytosine base editors (CBEs) guided by Cas9 and guide RNA can specifically convert a single C·G base pair to a T·A base pair. However, long-term persistence of CBEs in the cell can lead to unwanted off-target editing, compromising safety for therapeutic applications. Delivery of CBEs to a cell as ribonucleoprotein (RNP) complexes (as opposed to nucleic acid encoding constituents of a ribonucleoprotein complex) may allow temporal control of activity, but requires high levels of pure, active protein, which has proven challenging with current CBE variants. Moreover, many mutations remain inaccessible to editing due to the restricted editing window of current CBEs. There is therefore a need for cytosine base editors of high purity and with expanded or shifted editing windows that allow for editing of cytosines (Cs) at otherwise inaccessible positions.

### SUMMARY OF THE EMBODIMENTS

In accordance with one embodiment, the invention provides a fusion protein comprising, in an N to C direction, (i) an N-terminal domain of a Cas9 nickase (N-nCas9); (ii) a first linker sequence; (iii) a cytidine deaminase domain comprising SEQ ID NO:2; (iv) a second linker sequence; (v) a C-terminal domain of the Cas9 nickase (C-nCas9); and (vi) a uracil glycosylase inhibitor sequence (UGI).

The N-terminal domain of the Cas9 nickase may comprise an N-nCas9 amino acid sequence that is at least 85% identical to SEQ ID NO:1. In some embodiments, the N-terminal domain of the Cas9 nickase comprises SEQ ID NO:1. In other embodiments, the N-terminal domain of the Cas9 nickase consists of SEQ ID NO:1.

The C-terminal domain of the Cas9 nickase may comprise a C-nCas9 amino acid sequence that is at least 85% identical to SEQ ID NO:3. In some embodiments, the Cas9 nickase comprises SEQ ID NO:3. In other embodiments, the C-terminal domain of the Cas9 nickase consists of SEQ ID NO:3.

The uracil glycosylase inhibitor sequence may comprise a UGI amino acid sequence that is at least 85% identical to SEQ ID NO:4. In some embodiments, the uracil glycosylase inhibitor sequence comprises SEQ ID NO:4. In other embodiments, the uracil glycosylase inhibitor sequence consists of SEQ ID NO:4.

The first linker sequence may consist of 2 to 16 amino acid residues. In some embodiments, the first linker sequence is selected from the group consisting of Glycine-Serine, SEQ ID NO:5, and SEQ ID NO:6.

The second linker sequence may consist of 2 to 16 amino acid residues. In some embodiments, the second linker sequence is selected from the group consisting of Glycine-Serine, SEQ ID NO:5, and SEQ ID NO:6.

The fusion protein may comprise a nuclear localization signal sequence (NLS). In some embodiments, the nuclear localization signal sequence may be coupled to a terminus of the fusion protein selected from the group consisting of an N-terminus, a C-terminus, and combinations thereof. In some embodiments, the nuclear localization signal sequence is selected from the group consisting of SEQ ID NO:6, SEQ ID NO:7, and combinations thereof.

In some embodiments, the cytidine deaminase domain consists of SEQ ID NO:2.

The fusion protein may comprise SEQ ID NO:8. In some embodiments, the fusion protein consists of SEQ ID NO:8.

The fusion protein may comprise SEQ ID NO:9. In some embodiments, the fusion protein consists of SEQ ID NO:9.

In accordance with another embodiment, the invention provides a complex comprising a nucleic acid molecule and the fusion protein according to any one of the preceding claims. The nucleic acid molecule may be a single guide RNA.

In accordance with another embodiment, the invention provides a method of treating a subject having or suspected of having a disease or disorder, the method comprising administering the fusion protein ex vivo to a cell from the subject. The N-terminal domain of the Cas9 nickase and the C-terminal domain of the Cas9 nickase of the fusion protein may be bound to a single guide RNA.

In accordance with one embodiment, the invention provides a method of treating a subject having or suspected of having a disease or disorder, the method comprising administering the complex ex vivo to a cell from the subject.

In some embodiments, the single guide RNA comprises a sequence of at least 10 contiguous nucleotides that is complementary to at least 10 contiguous nucleotides of a target sequence, the target sequence comprising a mutation associated with the disease or disorder. In other embodiments the single guide RNA comprises a sequence of at least 18 contiguous nucleotides that is complementary to at least 18 contiguous nucleotides of a target sequence, the target sequence comprising a mutation associated with the disease or disorder.

In some embodiments, the method further comprising co-administering a uracil glycosylase inhibitor protein (UGIP) ex vivo to the cell from the subject. The uracil glycosylase inhibitor protein may comprise a UGIP amino acid sequence that is at least 85% identical SEQ ID NO:4. In some embodiments, the uracil glycosylase inhibitor protein comprises SEQ ID NO: 4.

The uracil glycosylase inhibitor protein may comprise a UGIP nuclear localization signal sequence (NLS). In some embodiments, the UGIP nuclear localization signal sequence is coupled to a terminus of the uracil glycosylase inhibitor protein selected from the group consisting of an N-terminus, a C-terminus, and combinations thereof. In some embodiments, the UGIP nuclear localization signal sequence is selected from the group consisting of SEQ ID NO:6, SEQ ID NO:7, and combinations thereof.

### BRIEF DESCRIPTION OF THE DRAWINGS

The foregoing features of embodiments will be more readily understood by reference to the following detailed description, taken with reference to the accompanying drawings, in which:

FIG. 1A is a schematic representation of eA3A-BE3 and an engineered base editor with 16 amino acid (a.a.) linker (CE_16_eA3A), in accordance with an embodiment of the invention. FIG. 1A discloses “SGGSSGGSSGSETPGTSESATPESSGGSSGGS” as SEQ ID NO: 20. FIG. 1B shows bar graphs showing C to T editing frequencies of CE_16_eA3A and eA3A-BE3 after RNP delivery at FANCF-M-b, PPP1R12C site3, and AAVS1. Each bar graph shows its respective target site sequence at the top of the bar graph. All editing frequencies were quantified by high-throughput sequencing (HTS) and are shown as means with error bars representing SD of n=3 biologically independent replicates on different days. Asterisks indicate statistically significant differences between eA3A-BE3 and CE_16_eA3A editing (****: P<0.0001; *: P<0.05; ns: P>0.05, not significant). All statistical testing was performed using two-way ANOVA. FIG. 1B disclosed SEQ ID NOS 21-23, respectively, in order of appearance.

FIG. 2A shows heat maps demonstrating average C-to-T base editing frequencies for eA3A_BE3, CE_16_eA3A, and CE_2_eA3A delivered as RNPs at 9 target sites in HEK293T cells. Each target site contains a preferential TC motif and one or more bystander cytidines within the protospacers, in accordance with embodiments of the invention. Positions with a cognate C preceded by a 5′ T (TC motifs) are indicated with black triangles. FIG. 2B shows average C-to-T base editing efficiencies in different contexts (TC (star), CC (circle), GC (square) and AC (triangle)) from the 9 target sites of FIG. 2A. Each data point represents the mean of triplicate measurements for each C in each target site. FIG. 2C is a schematic representation of the base editing window of CE_16_eA3A, CE_2_eA3A, and eA3A-BE3 with the heat map corresponding to the relative C to T editing activity at different positions of TC motifs within the protospacer regions. FIG. 2A discloses SEQ ID NOS 24-32, respectively, in order of appearance.

FIG. 3A shows bar graphs demonstrating dose-dependent base editing of C8, by CE_16_eA3A, CE_2_eA3A, and eA3A-BE3 at an AAVS1 target site, in accordance with embodiments of the invention. Edited nucleotides that have been converted from target C8 are: dark gray, bottom segment of each bar, C to T; light gray, middle segment of each bar, C to G; dark gray, top segment of each bar, C to A. FIG. 3B shows bar graphs demonstrating base editing of C8, by CE_16_eA3A, CE_2_eA3A, and eA3A-BE3 co-administered with varying doses of UGI-NLS at an AAVS1 target site. FIG. 3C is a bar graph showing that the addition of 5 molar equivalents of UGI-NLS protein to 2 μM CE_16_eA3A RNPs increased C to T editing from 43.1% to 85.0%. FIG. 3D is a bar graph showing indel formation after adding purified UGI-NLS to RNPs of CE_16_eA3A, CE_2_eA3A, and eA3A-BE3 at different ratios. Asterisks indicate statistically significant differences in editing efficiencies observed between untreated cells and cells treated with RNPs of CE_16_eA3A, CE_2_eA3A, or eA3A-BE3 and various ratio of purified UGI proteins. (ns: P>0.05, not significant; *: P<0.05; **: P<0.01; ****: P<0.0001). All statistical testing was performed using one-way ANOVA. FIG. 3A discloses SEQ ID NO: 23.

FIG. 4A shows plots comparing dose-dependent C base editing frequencies of BCL11A enhancer by CE_16_eA3A and eA3A_BE3 RNPs. Base edits were quantified by high-throughput sequencing. FIG. 4B shows plots of HbF levels of erythroid progeny after in vitro erythroid maturation from three healthy donors edited by different concentrations of CE_16_eA3A and eA3A_BE3 RNPs. HbF levels were quantified by HPLC analysis. FIG. 4C shows plots of correlation of HbF levels versus C editing rates of erythroid progeny differentiated from CD34⁺ HSPCs edited by 5-50 μM CE_16_eA3A or eA3A_BE3 RNPs. Circles, squares, or triangles represent an individual healthy donor. Mean values are indicated in bars. The various concentrations are shown with different colors. (Black: 0 μM, Dark gray: 5 μM, Light gray: 10 μM, White: 20 μM, Horizontally-hatched: 30 μM, Vertically-hatched: 40 μM, and Cross-hatched: 50 μM).

FIG. 5A shows two Coomassie-stained SDS-PAGE gels showing expression levels of CE_16_eA3A and eA3A-BE3 protein in E. coli, in the soluble (S) and insoluble (IS) fractions. FIG. 5B is a Coomassie-stained SDS-PAGE gel showing eluates of CE_16_eA3A and eA3A-BE3 protein purified by nickel affinity, mono S and Q ion exchange, and size exclusion columns next to protein lysates. FIG. 5C is a bar graph showing yield of purified CE_16_eA3A and eA3A-BE3 protein. Bars represent mean values, and error bars represent the SD of three independent replicates.

FIG. 6 is a bar graph showing indel formation by CE_16_eA3A and eA3A-after RNP delivery at 3 target sites (FANCF-M-b, PPP1R12C site3, and AAVS1). Indel levels represent the sum of all indel values in the editing window at each target site. Asterisks indicate statistically significant differences in indel levels observed between eA3A-BE3 and CE_16_e3A at each site (ns: P>0.05, not significant; *: P<0.05; **: P<0.01). All statistical testing was performed using two-way ANOVA.

FIG. 7A is a schematic representation of engineered CE-eA3A variants with linker lengths of 16 a.a., 7 a.a., and 2 a.a, in accordance with embodiments of the invention. FIG. 7B is a graph showing frequencies of C to T editing by CE-eA3A variants after RNP delivery on cytidines at AAVS1. FIG. 7B discloses SEQ ID NO: 32.

FIG. 8A shows heat maps representing on target and off-target editing frequencies at EMX1 site 1 and VEGFA site2. FIG. 8B shows bar graphs demonstrating the ratio of off-target to on-target C to T editing efficiencies. Each on/off-targeting efficiency was calculated by accumulating of all edited Cs in the editing window of on-target site or off-target sites. Bars show mean values and error bars the SD from three independent replicates. FIG. 8A discloses SEQ ID NOS 27, 33-35, 25, and 36-38, respectively, in order of columns.

FIG. 9 is a bar graph comparing viability of CD34+ HSPCs edited with various concentrations (5-50 μM) of CE_16_eA3A and eA3A-BE3 RNPs.

FIG. 10 is a bar graph comparing C editing rates of CE_16_eA3A and eA3A_BE3 (20 μM) complexed with 1620 gRNA in human CD34+ HSPCs from three independent healthy donors. C editing rates were measured with deep sequence analysis. Asterisks indicate statistically significant differences between CE_16_eA3A and eA3A-BE3 editing (****: P<0.0001; **: P<0.01; ns: P>0.05, not significant). All statistical testing was performed using two-way ANOVA. FIG. 10 discloses SEQ ID NO: 39.

FIG. 11 is a bar graph showing indel formation by different concentrations (5-50 μM) of CE_16_eA3A and eA3A-BE3 RNPs at the +58 BCL11A erythroid enhancer region in human CD34+ HSPCs. Asterisks indicate statistically significant differences in indel levels observed between eA3A-BE3 and CE_16_e3A at each concentration (ns: P>0.05, not significant; *: P<0.05). All statistical testing was performed using two-way ANOVA.

FIG. 12A is a graph of high-throughput sequencing data of 59 potential off-target sites within CD34⁺ HSPCs edited with 30 μM CE_16_eA3A RNPs and eA3A-BE3 RNPs complexed with 1620 gRNA. Negative controls (mock) are indicated by open circles. FIG. 12B is a bar graph of on-target and off-target C editing efficiency at OT1 by CE_16_eA3A RNPs and eA3A-bE3 RNPs complexed with 1620 gRNA.

FIG. 13A is a plot showing dose dependent editing rates with CE_2_eA3A coupled with sgRNA-1618 targeting BCL11A enhancer in CD34⁺ HSPCs by high-throughput sequencing analysis. FIG. 13B is a plot showing HbF levels by HPLC analysis of erythroid progeny after dose-response of CE_2_eA3A RNPs complexed with sgRNA-1618 after electroporation of HSPCs. FIG. 13C is a plot showing correlation of HbF levels versus C editing rates in erythroid cells differentiated from CE_2_eA3A RNP-edited CD34⁺ HSPCs. Circles, squares, or triangles represent an individual healthy donor. Mean values are indicated in bars. Various concentrations are shown with different colors. (Black: 0 μM, Dark gray: 5 μM, Light gray: 10 μM, White: 20 μM, Horizontally-hatched: 30 μM, Vertically-hatched: 40 μM, and Cross-hatched: 50 μM).

FIG. 14 is a graph of guide RNA(1618)-dependent off-target editing analysis of HSPCs edited with CE_2_eA3A RNPs. Using the CasOFFinder tool, 146 potential genomic off target sites with 3 or fewer mismatches relative to the on-target BCL11A enhancer sequence were identified. In human CD34⁺ HSPCs edited by 30 μM CE_2_eA3A RNPs, the C editing efficiency of each of these 146 sites was quantified by high-throughput sequencing. Negative controls (mock) are indicated by open circles.

FIG. 15 shows target site sequences for loci disclosed herein, in accordance with embodiments of the invention, Cytosine bases are shown underlined and protospacer adjacent motifs (PAMs) are shown in bold italics. FIG. 15 discloses SEQ ID NOS 32, 24-28, 40, 29, 31, and 41-42, respectively, in order of appearance.

FIG. 16 is a table of expression plasmids described herein, in accordance with embodiments of the invention.

FIG. 17A is a bar graph showing base editing in unfractionated BM after 16 weeks as compared to input HSPCs. Human CD34+ HSPCs from one healthy donor were electroporated with 30 μM of CE_16_eA3A RNPs. The treated HSPCs were infused into NBSGW mice and bone marrow (BM) was harvested 16 weeks after transplantation. FIG. 17B is a bar graph showing a comparison of human chimerism in mice receiving unedited or edited HSPCs. Human cells in BM from all mice were quantified by flow cytometry using an anti-human CD45 antibody. Each dot indicates one mouse recipient. FIG. 17C is a bar graph showing HbF induction quantified by HPLC in human erythroid cells from engrafted BM. FIG. 17D is a bar graph showing a comparison of the percentage of fetal (F) cells between the mock and edited groups. The percentage of fetal cells in engrafted erythroid cells was measured by flow cytometry using an anti-human HbF antibody. Data are plotted as mean±SD (n=4 mice receiving edited cells and n=3 mice receiving unedited cells).

FIG. 18A is a bar graph showing percentage of B cells 16 weeks after transplantation with unedited or edited HSPCs. FIG. 18B is a bar graph showing percentage of granulocytes 16 weeks after transplantation with unedited or edited HSPCs. FIG. 18C is a bar graph showing percentage of monocytes 16 weeks after transplantation with unedited or edited HSPCs. FIG. 18D is a bar graph showing percentage of HSPC human lineages 16 weeks after transplantation with unedited or edited HSPCs. FIG. 18E is a bar graph showing percentage of erythroid cells 16 weeks after transplantation with unedited or edited HSPCs. Data are plotted as mean±SD (n=4 mice receiving edited cells and n=3 mice receiving unedited cells).

### DETAILED DESCRIPTION OF SPECIFIC EMBODIMENTS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

Unless otherwise stated, all amino acid sequences are from N to C and all nucleic acid sequences are from 5′ to 3′. Nucleobase abbreviations used herein are: adenine (A), cytosine (C), guanine (G), thymine (T), and uracil (U).

The term “deaminase” or “deaminase domain,” as used herein, refers to a protein or enzyme that catalyzes a deamination reaction. In some embodiments, the deaminase or deaminase domain is a cytidine deaminase, catalyzing the hydrolytic deamination of cytidine or deoxycytidine to uridine or deoxyuridine, respectively. In some embodiments, the deaminase or deaminase domain is a cytidine deaminase domain, catalyzing the hydrolytic deamination of cytosine to uracil. In some embodiments, the cytidine deaminase domain comprises SEQ ID NO:2. In some embodiments, the cytidine deaminase domain comprises a TC-specific APOBEC3A cytidine deaminase (Nat Biotechnol. 2018, 36, 977-982. PMID: 30059493). In other embodiments, the cytidine deaminase domain comprises a CC-specific APOBEC3G cytidine deaminase or A3G cytidine deaminase (Sci Adv. 2020, 6, eaba1773, PMID: 32832622). In some embodiments, the cytidine deaminase domain comprises a ZC-specific APOBEC1 cytidine deaminase, wherein Z is T, A, or C (Nature, 533, 2016, 420-424, PMID: 27096365). In other embodiments, the cytidine deaminase domain comprises an NC-specific evoAPOBEC1, CDA1, evoCDA1, FERNY, or evoFERNY cytidine deaminase, wherein N is A, T, G, or C (Nat Biotechnol. 2019, 37, 1070-1079. PMID: 31332326). In some embodiments, the cytidine deaminase domain comprises a cytidine deaminase with reduced Cas9-independent deamination activity, for example an APOBEC1 cytidine deaminase having one of the following amino acid substitutions: (1) W90Y+R126E, (2) W90Y+R132E, (3) R126E+R132E, (4) W90Y+R126E+R132E, (5) R33A, or (6) R33A+K34A (Nat Biotechnol, 2020, 38, 620-628, PMID: 32042165; Nature, 2019, 569, 433-437. PMID: 30995674). In some embodiments, the deaminase or deaminase domain is a naturally-occurring deaminase from an organism, such as a human, chimpanzee, gorilla, monkey, cow, dog, rat, or mouse. In some embodiments, the deaminase or deaminase domain is a variant of a naturally-occurring deaminase from an organism that does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase from an organism. U.S. Pat. No. 11,542,496 is hereby incorporated by referenced for its disclosure of deaminases, including cytidine deaminases.

The term “base editor (BE)” refers to an agent comprising a polypeptide that is capable of making a modification to a base (e.g., A, T, C, G, or U) within a nucleic acid sequence (e.g., DNA or RNA). In some embodiments, the base editor is capable of deaminating a base within a nucleic acid. In some embodiments, the base editor is capable of deaminating a base within a DNA molecule. In some embodiments, the base editor is a “cytosine base editor” (CBE). In some embodiments, a “cytosine base editor” is capable of deaminating a cytosine (C) in DNA. In some embodiments, the base editor is a protein (e.g., a fusion protein) comprising a nucleic acid programmable DNA binding protein (napDNAbp) fused to a cytidine deaminase. In some embodiments, the base editor comprises a napDNAbp, a cytidine deaminase, and a uracil glycosylase inhibitor (UGI). In some embodiments, the UGI comprises SEQ ID NO:4. U.S. Pat. No. 11,542,496 is hereby incorporated by referenced for its disclosure of base editors and base editing, including cytosine base editors and cytosine base editing.

The term “Cas9” or “Cas9 domain” refers to an RNA-guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casn1 nuclease or a CRISPR (clustered regularly interspaced short palindromic repeat)-associated nuclease. CRISPR is an adaptive immune system that provides protection against mobile genetic elements (viruses, transposable elements and conjugative plasmids) CRISPR clusters contain spacers, sequences complementary to antecedent mobile elements, and target invading nucleic acids CRISPR clusters are transcribed and processed into CRISPR RNA (crRNA). In type II CRISPR systems correct processing of pre-crRNA requires a trans-encoded small RNA (tracrRNA), endogenous ribonuclease 3 (me) and a Cas9 protein. The tracrRNA serves as a guide for ribonuclease 3-aided processing of pre-crRNA. Subsequently, Cas9/crRNtAtracrRNA endonucleolytically cleaves linear or circular dsDNA target complementary to the spacer. The target strand, which is not complementary to crRNA (and not complementary to a guide sequence of a sgRNA), is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species See, e.g., Jinek M., Chylinski K., Fonfara I., Hauer M., Doudna J. A., Charpentier E. Science 37816-821(2012), the entire contents of which is hereby incorporated by reference. Cas9 recognizes a short motif in the CRISPR repeat sequences (the PAM or protospacer adjacent motif) to help distinguish self versus non-self Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S, Lai H S., Lin S P., Qian Y., Jia H. G., Najar F. Z., Ren Q., Zhu H, Song L, White J, Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E, Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski. Rhun, and Charpentier, “The tracrRNA and Cas9 families of type II CRISPR-Cas immunity systems” (2013) RNA Biology 10.5, 726-737; the entire contents of which are incorporated herein by reference. In some embodiments, a Cas9 nuclease has an inactive (e.g., an inactivated) DNA cleavage domain, that is, the Cas9 is a nickase. U.S. Pat. No. 11,542,496 is hereby incorporated by referenced for its disclosure of Cas9.

In some embodiments, Cas9 refers to Cas9 protein from: Streptococcus pyogenes (NCBI Ref: NC_002737.2) (SpCas9); Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref. NC 021314.1); Belliella baltica (NCBI Ref. NC_018010.1); Psychroflexus torquisl (NCBI Ref: NC_018721.1); Streptococcus thermophilus (NCBI Ref: YP_820832.1); Listeria innocua (NCBI Ref: NP_472073.1); Campylobacter jejuni (NCBI Ref. YP_002344900 1); or Neisseria meningitidis (NCBI Ref: YP_002342100.1), Staphylococcus aureus (NCBI Ref: AYD60528.1). In some embodiments, Cas9 refers to a SpCas9 variant with a PAM requirement that is not NGG (where N is A, C, G, or T), including, but not limited to: (1) SpCas9-VQR(NGA) (Nature, 2015. 523, 481-5, PMID: 26098369), (2) SpCas9-NG(NG) (Science, 2018, 361, 1259-1262, PMID: 30166441). (3) SpCas9-NRNH(NRNH) (Nat Biotechnol, 2020, 38, 471-481, PMID. 32042170), (4) SpG(NG) or (5) SpRY (NR>NY) (Science, 2020, 368, 290-296, PMID: 32217751). In some embodiments, Cas9 refers to a high fidelity SpCas9 variant, including, but not limited to: (1) eSpCas9 (Science. 2016, 351, 84-88, PMID: 26628643), (2) SpCas9-HF1 (Nature, 2016, 529, 490-5, PMID: 26735016), (3) HypaSpCas9 (Nature. 2017, 550, 407-410, PMID: 28931002), (4) HeFSpCas9 (Genome Biol. 2017, 18, 190, PMID. 28985763), (5) HiFi Cas9 (Nat Med 2018, 24, 1216-1224, PMID: 30082871), (6) Sniper (Nat Commun. 2018, 9, 3048, PMID: 30082838), (7) evoSpCas9 (Nat Biotechnol. 2018, 36, 265-271. PMID. 29431739), (8) Blackjack, eSpCas9-plus, and SpCas9-HF1-plus (Nat Commun. 2020, 11, 1223, PMID: 32144253), or (9) SuperFi-Cas9 (Nature. 2022, 603, 343-347. PMID: 35236982).

The term “Cas9 nickase” (nCas9) as used herein, refers to a Cas9 protein, or fragment thereof, that is capable of cleaving only one strand of a duplexed nucleic acid molecule (e.g., a duplexed DNA molecule comprising a targeted strand and a non-targeted strand). In some embodiments, a Cas9 nickase has an active HNH nuclease domain and is able to cleave the non-targeted strand of DNA, i.e., the strand bound by a gRNA. Further, such a Cas9 nickase has an inactive RuvC nuclease domain and is not able to cleave the targeted strand of the DNA, i.e., the strand where base editing is desired. In some embodiments, a SpCas9 nickase is a naturally occurring SpCas9 protein having a D10A amino acid substitution. As used herein, “target sequence” refers to a sequence of a non-targeted strand of a duplexed DNA molecule, i.e., the strand bound by a gRNA, the target sequence being complementary to a guide sequence of the gRNA. Base editing occurs at a target site of a targeted strand of the duplexed DNA molecule, opposite the target sequence of the non-targeted strand. In some embodiments, the target site comprises a canonical PAM sequence (NGG). U.S. Pat. No. 11,542,496 is hereby incorporated by referenced for its disclosure of Cas9 nickases.

In some embodiments, the napDNAbp of the base editor is a nCas9 domain. In some embodiments, the base editor comprises a nCas9 domain fused to a cytidine deaminase. In some embodiments, the nCas9 domain is an N-terminal domain of a Cas9 nickase (N-nCas9). As used herein, an “N-terminal domain of a Cas9 nickase” (N-nCas9) refers to an N-terminal portion of a Cas9 nickase, the N-terminal portion of the Cas9 nickase comprising (i) a RuvCI domain, (ii) a BH domain, (iii) a REC domain, (iv) a RuvCII domain, (v) a HNH domain, and (vi) an N-terminal portion of a RuvCIII domain of the Cas9 nickase, and lacking (vii) a C-terminal portion of the RuvCIII domain and (viii) a PI domain of the Cas9 nickase. In some embodiments, the N-terminal domain of a Cas9 nickase comprises an N-nCas9 amino acid sequence that is at least 85% identical to SEQ ID NO:1. In some embodiments, the N-terminal domain of a Cas9 nickase comprises SEQ ID NO: 1. In some embodiments, the nCas9 domain is a C-terminal domain of a Cas9 nickase (C-nCas9). As used herein, an “C-terminal domain of a Cas9 nickase” (C-nCas9) refers to an C-terminal portion of a Cas9 nickase, the C-terminal portion of the Cas9 nickase comprising (i) a C-terminal portion of a RuvCIII domain and (ii) a PI domain of the Cas9 nickase, and lacking (iii) a RuvCI domain, (iv) a BH domain, (v) a REC domain, (vi) a RuvCII domain, (vii) a HNH domain, and (viii) an N-terminal portion of the RuvCIII domain of the Cas9 nickase. In some embodiments, the C-terminal domain of a Cas9 nickase comprises a C-nCas9 amino acid sequence that is at least 85% identical to SEQ ID NO:3. In some embodiments, the C-terminal domain of a Cas9 nickase comprises SEQ ID NO:3. As used herein, “portion” means a part of a whole and not an entirety of the whole.

The term “linker,” as used herein, refers to a bond (e.g., covalent bond), chemical group, or a molecule linking two molecules or moieties, e.g., two domains of a fusion protein, such as, for example, a Cas9 domain and a nucleic acid-editing domain (e.g., a cytidine deaminase). In some embodiments, a linker comprises an amino acid sequence. In some embodiments, the linker is 2-100 amino acids in length, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. In some embodiments, a linker comprises the amino acid sequence SGSETPGTSESATPES (SEQ ID NO: 5), which may also be referred to as the XTEN linker. In some embodiments, a linker comprises the amino acid sequence GS. In some embodiments, a linker comprises a nuclear localization signal sequence. Additional linkers are disclosed in U.S. Pat. No. 11,542,496, which is hereby incorporated by reference for its disclosure of linkers.

The term “nuclear localization sequence,” “nuclear localization signal,” “nuclear localization signal sequence,” or “NLS” refers to an amino acid sequence that promotes import of a protein into the cell nucleus, for example, by nuclear transport. Nuclear localization sequences are known in the art and would be apparent to the skilled artisan. In some embodiments, the NLS is a monopartite NLS. In some embodiments, the NLS is a bipartite NLS. Bipartite NLSs are separated by a relatively short spacer sequence (e.g., from 2-20 amino acids, from 5-15 amino acids, or from 8-12 amino acids). For example, NLS sequences are described in U.S. Pat. No. 11,542,496; Plank et al., international PCT application, PCT/EP2000/011690, filed Nov. 23, 2000, published as WO/2001/038547 on May 31, 2001; and Kethar, K. M. V., et al., “Application of bioinformatics-coupled experimental analysis reveals a new transport-competent nuclear localization signal in the nucleoptotein of Influenza A virus strain” BMC Cell Biol, 2008, 9: 22; the contents of each of which are incorporated herein by reference for their disclosure of exemplary nuclear localization sequences. In some embodiments, a NLS comprises the amino acid sequence of SEQ ID NO: 6, SEQ ID NO:10, SEQ ID NO:11, SEQ ID NO:12, or SEQ ID NO:13. U.S. Pat. No. 11,542,496 is hereby incorporated by referenced for its disclosure of nuclear localization sequences.

The term “nucleic acid programmable DNA binding protein” or “napDNAbp” refers to a protein that associates with a nucleic acid (e.g., DNA or RNA), such as a guide nucleic acid, that guides the napDNAbp to a specific nucleic acid sequence. For example, a nCas9 protein can associate with a guide RNA that guides the nCas9 protein to a specific DNA sequence that has complementary to the guide RNA. U.S. Pat. No. 11,542,496 is hereby incorporated by referenced for its disclosure of napDNAbps.

The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) portion of the fusion protein. A protein may comprise different domains, for example, a nucleic acid binding domain (e.g., the gRNA binding domain of Cas9 that directs the binding of the protein to a target site) and a nucleic acid cleavage domain or a catalytic domain of a nucleic-acid editing protein. U.S. Pat. No. 11,542,496 is hereby incorporated by referenced for its disclosure of fusion proteins.

The fusion proteins disclosed herein form a complex with (e.g., bind or associate with) one or more RNA(s) that is not a target for cleavage. In some embodiments, an RNA-programmable nuclease, when in a complex with an RNA, may be referred to as a ribonucleoprotein complex, ribonucleoprotein, RNP, or RNP complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs). Typically, gRNAs that exist as single RNA species comprise two domains: (1) a domain that shares homology to a target nucleic acid (e.g., and directs binding of a Cas9 complex to the target); and (2) a domain that binds a Cas9 protein. In some embodiments, domain (2) corresponds to a sequence known as a tracrRNA, and comprises a stem-loop structure. See, e.g., Jinek et al., Science 337:816-821(2012). U.S. Pat. No. 11,542,496 is hereby incorporated by referenced for its disclosure of ribonucleoproteins and gRNAs, including sgRNAs.

In some embodiments, the guide RNA is from 15-100 nucleotides long and comprises a sequence of at least 10 contiguous nucleotides that is complementary to a target sequence. In some embodiments, the guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long. In some embodiments, the guide RNA comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target sequence. In some embodiments, the target sequence is a DNA sequence. In some embodiments, the target sequence is a sequence in the genome of a mammal. In some embodiments, the target sequence is a sequence in the genome of a human. In some embodiments, the guide nucleic acid (e.g., guide RNA) is complementary to a sequence associated with a disease or disorder. In some embodiments, the guide nucleic acid (e.g., guide RNA) is complementary to a sequence associated with a disease or disorder

In some embodiments, the guide RNA comprises a structure 5′-[guide sequence]-guuuuagagcuagaaauagcaaguuaaaauaaaggcuaguccguuaucaacuugaaaaaguggcaccgagucggugcuu uuu-3′ (SEQ ID NO: 14), wherein the guide sequence comprises a sequence that is complementary to the target sequence. In other embodiments, the guide RNA comprises a structure 5′-[guide sequence]-guuuuagagcuagaaauagcaaguuaaaauaaggcuaguccguuaucaacuugaaaaaguggcaccgagucggugcuuu u-3′ (SEQ ID NO:15). The guide sequence is typically 20 nucleotides long. The sequences of suitable guide RNAs for targeting Cas-embedded cytosine base editor proteins to specific genomic target sites will be apparent to those of skill in the art. See, e.g., U.S. Pat. No. 11,542,496, which is incorporated by reference herein in its entirety.

“Cas-embedded cytosine base editor” (CE_CBE), “Cas-embedded CBE,” and the like, as used herein, means a fusion protein comprising, in an N to C direction, a N-nCas9, a cytidine deaminase domain, a C-nCas9, and a UGI. In some embodiments, the cytidine deaminase domain is coupled to the N-nCas9 by a first linker, and coupled to the C-nCas9 by a second linker. In some embodiments, the first linker is the same as the second linker. In some embodiments, the first linker is different than the second linker. In some embodiments, the CE_CBE comprises an N-terminal NLS. In some embodiments, the CE_CBE comprises as C-terminal NLS. In some embodiments, the CE_CBE comprises an N-terminal NLS and a C-terminal NLS. As used herein, “CE eA3A” refers to a CE_CBE wherein the cytidine deaminase domain comprises SEQ ID NO:2. CE_eA3As include, but are not limited to, CE_16_eA3A (CE_16_16_eA3A), CE_7_16_eA3A, CE_2_16_eA3A, CE_2_7_eA3A, and CE_2_2_eA3A (CE_2_eA3A), disclosed herein.

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development.

“Complementary” it is meant that a nucleic acid (e.g. RNA) comprises a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes; adenine (A) pairing with thymidine (T), adenine (A) pairing with uracil (U), and guanine (G) pairing with cytosine (C) [DNA, RNA]. In addition, it is also known in the art that for hybridization between two RNA molecules (e.g., dsRNA), guanine (G) base pairs with uracil (U). For example, G/U base-pairing is partially responsible for the degeneracy (i.e., redundancy) of the genetic code in the context of tRNA anti-codon base-pairing with codons in mRNA. In the context of this disclosure, a guanine (G) of a protein-binding segment (dsRNA duplex) of a subject DNA-targeting RNA molecule is considered complementary to a uracil (U), and vice versa. As such, when a G/U base-pair can be made at a given nucleotide position a protein-binding segment (dsRNA duplex) of a subject DNA-targeting RNA molecule, the position is not considered to be non-complementary, but is instead considered to be complementary. U.S. Publication No. US 2019/0010520 is hereby incorporated by reference for its disclosure of complementarity (including “complementary”) and hybridization (including “hybridizable”).

The term “target site” or “target site sequence” refers to a sequence of a targeted strand of a duplexed nucleic acid molecule that is modified by a base editor, such as a fusion protein comprising a cytidine deaminase, e.g., a CE_CBE.

The terms “treatment,” “treat,” and “treating,” refer to a clinical intervention aimed to reverse, alleviate, delay the onset of, or inhibit the progress of a disease or disorder, or one or more symptoms thereof.

Cytosine base editors (CBEs) convert C·G base pairs to T·A base pairs within a small editing window specified by a single guide RNA (sgRNA), allowing for single base changes without generating double strand breaks (Komor, Kim et al. 2016, Nishida, Arazoe et al. 2016, Rees and Liu 2018). CBEs are constructed from a cytidine deaminase domain fused to a Cas9 nickase (nCas9). Compared to the CRISPR-Cas9 gene editing system, CBEs are more efficient and carry less risk of genomic alterations such as insertions, deletions, and translocations (Molla and Yang 2019) and have therefore emerged as a popular tool for specific editing of single nucleotide variants (SNVs) in the genomes of diverse organisms (Huang, Newby et al. 2021). However, major limitations stand in the way of CBEs reaching their full potential as research and therapeutic tools, including off-target editing and challenges with ribonucleoprotein (RNP) delivery. Moreover, the editing window of recombinant CBE proteins is restricted, generally, to 4-5 nucleotides [positions 4-8(or 9)] within a protospacer (Zeng, Wu et al. 2020, Jang, Jo et al. 2021); together with protospacer adjacent motif (PAM) requirements of Cas9 targeting. Due to these constraints, many genetic variants of interest cannot be effectively targeted with current CBE technologies.

Multiple strategies have been developed to reduce off-target editing that can occur either due to improper targeting of the CBE to erroneous genomic loci by its Cas9:sgRNA component, or by indiscriminate activity of the cytidine deaminase domain (Rees and Liu 2018, Grunewald, Zhou et al. 2019, Jin, Zong et al. 2019, Zhou, Sun et al. 2019, Zuo, Sun et al. 2019). As such, high-fidelity Cas9 variants and cytidine deaminase variants with enhanced DNA specificity have been reported to decrease off-target events of CBEs (Rees, Komor et al. 2017, Gehrke, Cervantes et al. 2018, Doman, Raguram et al. 2020, Yu, Leete et al. 2020). The engineering of Cas-embedded CBEs (CE_CBEs) reduces off-target editing by inserting or embedding a cytidine deaminase domain into the middle of a Cas9 ORF, bringing the cytidine deaminase active site closer to the target ssDNA loop compared to previous CBE constructs with an N-terminally linked deaminase (Liu, Zhou et al. 2020).

Small-molecule controllable CBE systems (such as rapamycin-inducible CBE) can improve specificity by temporally controlling its cellular activity (Berrios, Evitt et al. 2021, Long, Liu et al. 2021). Long-term persistence of CBEs can also be minimized by delivery in the form of CBE:sgRNA RNP complexes (Zuris, Thompson et al. 2015, Rees, Komor et al. 2017, Anzalone, Koblan et al. 2020) (Zeng, Wu et al. 2020) which are degraded by endogenous cellular proteases (Kim, Kim et al. 2014, Jang, Jo et al. 2021). However, wider use of CBE RNPs for biomedical applications has been hampered due to the difficulty of producing high-quality CBE protein preparations (Jang, Jo et al. 2021).

Recombinant CBE RNPs with high purity/yield, high editing efficiency, and varied editing windows are needed for optimal safety, delivery, and activity toward a wider range of human disease-associated SNVs.

In some embodiments, disclosed herein are CE_CBEs with optimized linker lengths between an nCas9 domain and an eA3A cytidine deaminase domain with reduced off-target DNA/RNA editing activities for base editing via protein delivery and having improved protein yield and solubility compared to N-terminal linking of eA3A to nCas9. In some embodiments, a CE_CBE disclosed herein (also referred to as CE_eA3A, a CE_eA3A variant, CE_eA3A fusion protein, and the like) is a fusion protein comprised of, in an N (amino-terminus) to C (carboxy-terminus) direction, (i) an N-terminal domain of a Cas9 nickase (N-nCas9); (ii) a first linker sequence; (iii) a cytidine deaminase domain; (iv) a second linker sequence; (v) a C-terminal domain of the Cas9 nickase (C-nCas9); and (vi) a uracil glycosylase inhibitor sequence (UGI). The N-terminal domain of the Cas9 nickase may comprise an N-nCas9 amino acid sequence that is at least 85% identical to SEQ ID NO:1. In some embodiments, the N-terminal domain of the Cas9 nickase comprises SEQ ID NO:1. In some embodiments, the N-terminal domain of the Cas9 nickase consists of SEQ ID NO:1. In some embodiments, the cytidine deaminase domain comprises eA3A cytidine deaminase (SEQ ID NO:2). In some embodiments, the cytidine deaminase domain consists of eA3A cytidine deaminase (SEQ ID NO:2).

The C-terminal domain of the Cas9 nickase may comprise an C-nCas9 amino acid sequence that is at least 85% identical to SEQ ID NO:3. In some embodiments, the C-terminal domain of the Cas9 nickase comprises SEQ ID NO:3. In some embodiments, the C-terminal domain of the Cas9 nickase consists of SEQ ID NO:3.

The uracil glycosylase inhibitor sequence may comprise a UGI amino acid sequence that is at least 85% identical to SEQ ID NO:4. In some embodiments, the uracil glycosylase inhibitor sequence comprises SEQ ID NO:4. In some embodiments, the uracil glycosylase inhibitor sequence consists of SEQ ID NO:4.

In some embodiments, the first linker sequence consists of 2 to 16 amino acid residues. In some embodiments, the first linker sequence is 2-100 amino acids in length, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. The first linker sequence may be selected from the group consisting of Glycine-Serine, SEQ ID NO:5, SEQ ID NO:6, SEQ ID NO:7, SEQ ID NO:10, SEQ ID NO: 11, SEQ ID NO: 12, SEQ ID NO:13, and other suitable linkers disclosed herein or as known in the art, including, but not limited to, a nuclear localization signal sequence, as disclosed herein or as known in the art.

In some embodiments, the second linker sequence consists of 2 to 16 amino acid residues. In some embodiments, the second linker sequence is 2-100 amino acids in length, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. The second linker sequence may be selected from the group consisting of Glycine-Serine, SEQ ID NO:5, SEQ ID NO:6, SEQ ID NO:7, SEQ ID NO:10, SEQ ID NO:11, SEQ ID NO: 12, SEQ ID NO: 13, and other suitable linkers disclosed herein or as known in the art, including, but not limited to, a nuclear localization signal sequence, as disclosed herein or as known in the art.

In some embodiments, the fusion protein comprises a nuclear localization signal sequence (NLS). The nuclear localization signal sequence may be coupled to a terminus of the fusion protein selected from the group consisting of an N-terminus, a C-terminus, and combinations thereof. In some embodiments, the nuclear localization signal sequence is selected from the group consisting of SEQ ID NO:6, SEQ ID NO:7, SEQ ID NO:10, SEQ ID NO: 11, SEQ ID NO: 12, SEQ ID NO:13, other nuclear localization signal sequences, as disclosed herein or as known in the art, and combinations thereof.

In some embodiments, the fusion protein comprises a sequence selected from the group consisting of SEQ ID NO:8 and SEQ ID NO:9. In some embodiments, the fusion protein consists of a sequence selected from the group consisting of SEQ ID NO:8 and SEQ ID NO:9.

Some aspects of this disclosure provide a ribonucleoprotein, the ribonucleoprotein being a complex comprising a CE_eA3A fusion protein and a nucleic acid molecule (also referred to as a CE_eA3A RNP, CE_eA3A RNP variant, and the like). The nucleic acid molecule may be a guide RNA (gRNA), such as a single guide RNA (sgRNA), the sgRNA comprising a guide sequence.

CE_eA3A variants disclosed herein display an expanded C-to-T editing window thereby permitting access to previously inaccessible positions in a target nucleic acid sequence. In some embodiments, CE_eA3A RNP variants disclosed herein display specific and highly efficient C-to-T editing with shifted or expanded editing windows in human cells, thereby significantly increasing target site sequence space.

Several strategies have been previously applied to alter the editing window of CBEs, such as combining natural or modified cytosine deaminases with different Cas proteins, fusing cytosine deaminases to circularly permuted Cas9 variants, or using stiff, proline-rich linkers of specific lengths between Cas9 and APOBEC1 (Huang, Zhao et at. 2019, Tan, Zhang et al. 2019, Thuronyi, Koblan et al. 2019). These engineered CBEs were introduced to cells via plasmid delivery, and the feasibility of their delivery as RNP complexes has not yet been characterized. CE_eA3A fusion proteins and RNP complexes disclosed herein represent the first demonstration of stable and efficient CBE RNPs with expanded or shifted editing windows, which can enable editing Cs at otherwise inaccessible positions.

To quantify this, among 1517 human pathogenic single nucleotide variants (SNVs) within the BEable-GPS (Base Editable prediction of Global Pathogenic-related SNVs) database (Gehrke, Cervantes et at. 2018, Wang, Gao et al. 2019), we identified 143 SNVs (~9%) correctable by eA3A-BE3, but strikingly 229 SNVs (~15%) correctable by CE_16_eA3A and 176 SNVs (~12%) correctable by CE_2_eA3A. Our analysis points to 86 SNVs outside the editing window of eA3A-BE3 that are predicted to be correctable by at least one CE_eA3A fusion protein or RNP complex thereof disclosed herein, strongly supporting their ability to expand the target site sequence space of base editors for future biomedical applications.

In other embodiments, CE_eA3A RNPs disclosed herein may be delivered to a cell in trans with a uracil glycosylase inhibitor protein (UGIP), resulting in increased C-to-T editing efficiency and target purity in a dose-dependent manner and with minimal indel formation. In some embodiments, the uracil glycosylase inhibitor protein comprises a UGIP amino acid sequence that is at least 85% identical SEQ ID NO:4. In some embodiments, the uracil glycosylase inhibitor protein comprises SEQ ID NO:4. In some embodiments, the uracil glycosylase inhibitor protein comprises SEQ ID NO:4. The uracil glycosylase inhibitor protein may comprise a UGIP nuclear localization signal sequence (NLS). In some embodiments, the UGIP nuclear localization signal sequence is coupled to a terminus of the uracil glycosylase inhibitor protein selected from the group consisting of an N-terminus, a C-terminus, and combinations thereof. In some embodiments, the UGIP nuclear localization signal sequence is selected from the group consisting of SEQ ID NO:6, SEQ ID NO:7, SEQ ID NO:10, SEQ ID NO:11, SEQ ID NO: 12, SEQ ID NO:13, other nuclear localization signal sequences, as disclosed herein or as known in the art, and combinations thereof.

In some embodiments, a method is provided for effectively editing a target site sequence of a therapeutically relevant genomic locus by administering a CE_eA3A fusion protein disclosed herein to a cell ex vivo. A CE_eA3A fusion protein may be administered as a RNP in complex with a nucleic acid molecule, such as a gRNA, e.g., a sgRNA. In some embodiments, the therapeutically relevant locus is BCL11A erythroid enhancer, which is associated with sickle cell disease in human hematopoietic stem and progenitor cells (HSPCs). In some embodiments, a CE_eA3A fusion protein or a RNP complex comprising a CE_eA3A fusion protein in complex with a nucleic acid molecule, such as a gRNA, e.g., a sgRNA is administered to a cell via electroporation.

The gRNA, e.g., a sgRNA, may comprise a sequence of at least 10 contiguous nucleotides that is complementary to at least 10 contiguous nucleotides of a target sequence, the target sequence comprising a mutation associated with the disease or disorder. In some embodiments, the gRNA, e.g., a sgRNA, comprises a sequence of at least 10 contiguous nucleotides that is perfectly complementary to at least 10 contiguous nucleotides of a target sequence, the target sequence comprising a mutation associated with the disease or disorder.

In some embodiments, the gRNA, e.g., a sgRNA, comprises a sequence of at least 18 contiguous nucleotides that is complementary to at least 18 contiguous nucleotides of a target sequence, the target sequence comprising a mutation associated with the disease or disorder. In some embodiments, the gRNA, e.g., a sgRNA, comprises a sequence of at least 18 contiguous nucleotides that is perfectly complementary to at least 18 contiguous nucleotides of a target sequence, the target sequence comprising a mutation associated with the disease or disorder.

In some embodiments, the gRNA, e.g., a sgRNA, comprises a sequence of at least 20 contiguous nucleotides that is complementary to at least 20 contiguous nucleotides of a target sequence, the target sequence comprising a mutation associated with the disease or disorder. In some embodiments, the gRNA, e.g., a sgRNA, comprises a sequence of at least 20 contiguous nucleotides that is perfectly complementary to at least 20 contiguous nucleotides of a target sequence, the target sequence comprising a mutation associated with the disease or disorder.

### Example 1: Cas-Embedded Base Editor RNPs Display High Activity

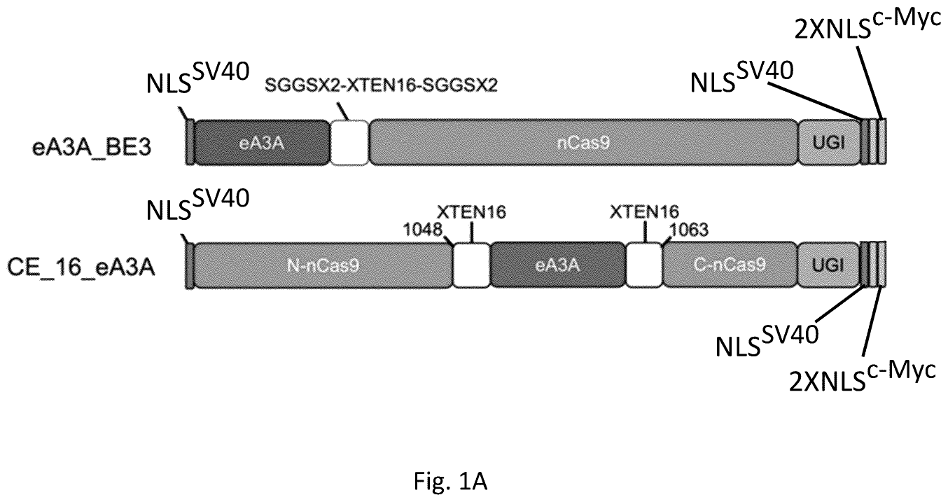

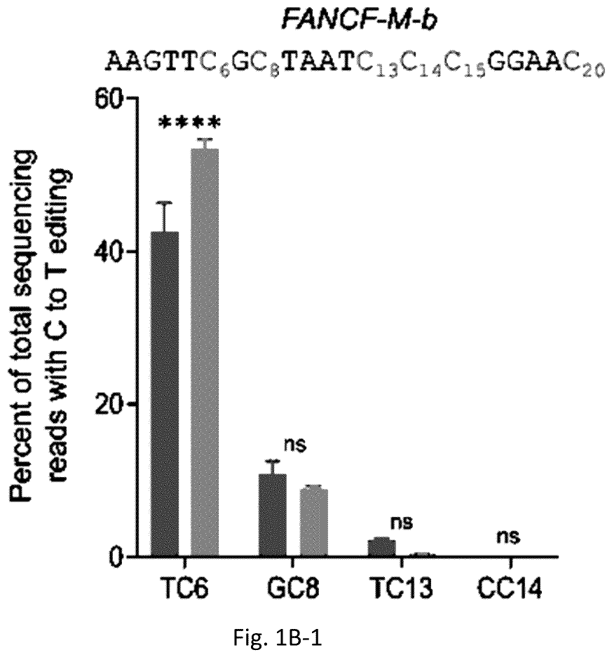

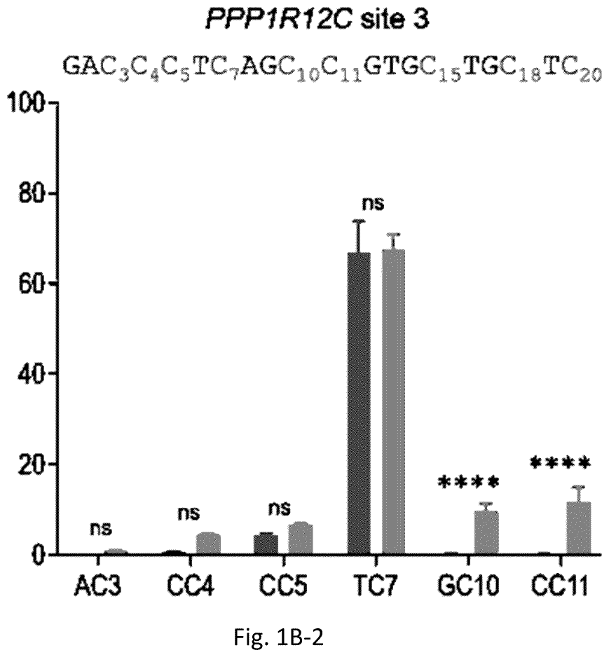

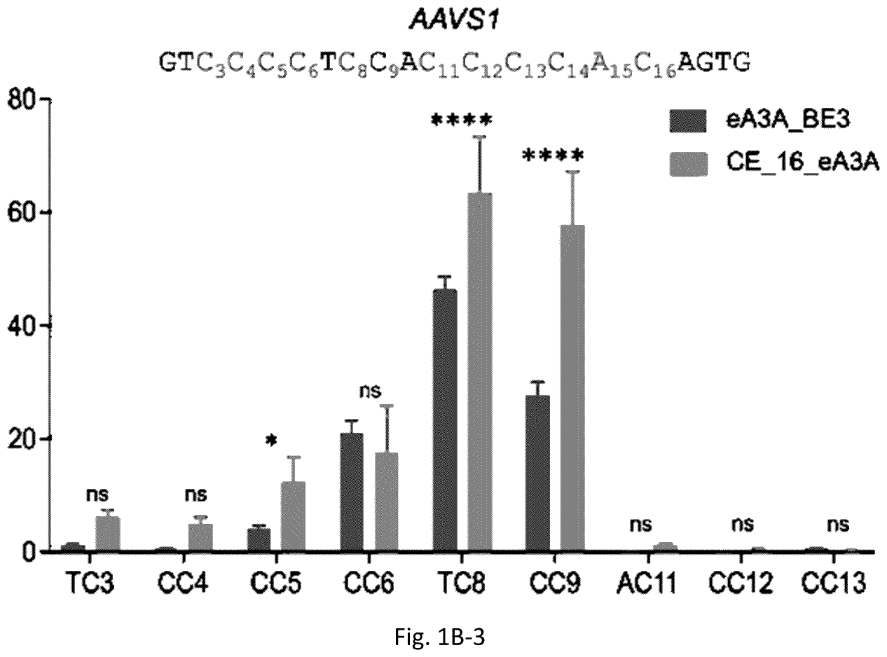

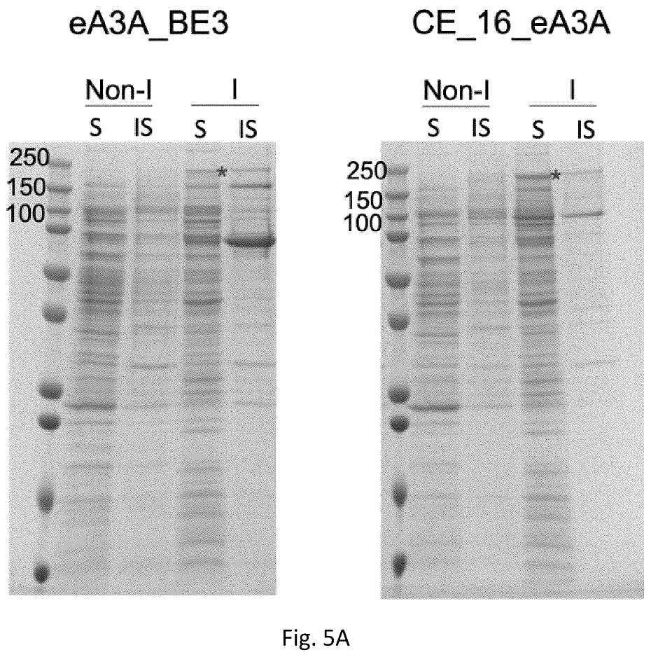

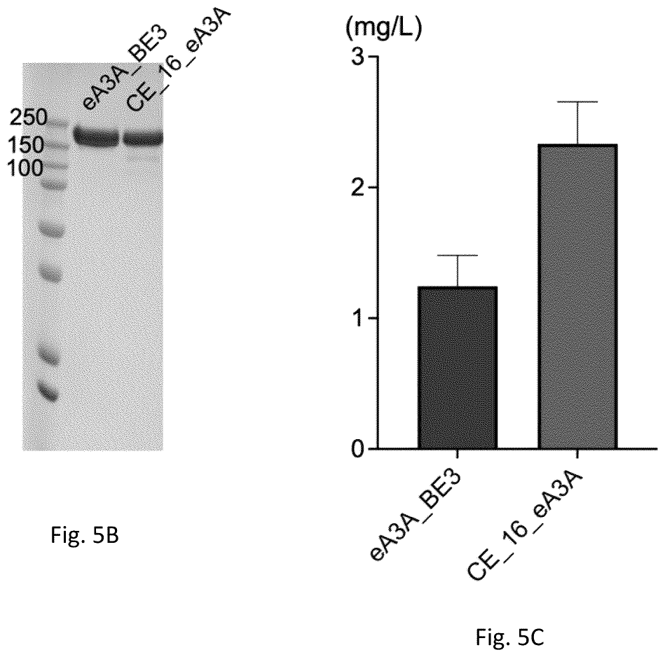

To construct Cas-embedded (CE) versions of the third-generation base editor BE3, we examined previously described tolerant sites of Cas9 for domain insertion (Oakes, Nadler et al. 2016, Liu, Zhou et al. 2020, Chu, Packer et al. 2021) and chose the 1048Thr-1063Ile region based on its proximity to the single stranded target DNA loop that is the substrate for base editing (FIG. 1A). Deaminase insertions in this region have resulted in effective base editors when introduced to cells via DNA vector delivery (Liu, Zhou et al. 2020). For the cytidine deaminase domain, we selected the single-domain cytosine base editor eA3A (SEQ ID NO:2), which has a strong TC motif preference, minimized sgRNA-independent and -dependent off-target DNA editing, and reduced off-target RNA editing activity compared to rat APOBEC1 CBEs (Gehrke, Cervantes et al. 2018). eA3A (SEQ ID NO:2) was inserted into the Cas9 ORF flanked by XTEN linkers (16 a.a.) at both sides. We also added nuclear localization signal (NLS) sequences at the C-terminus to enhance the editing efficiency (Suzuki, Tsunekawa et al. 2016, Koblan, Doman et al. 2018) (Zafra, Schatoff et al. 2018) (Wu, Zeng et al. 2019). The resulting base editor fusion protein was named CE_16_eA3A (SEQ ID NO:8). In addition, we generated eA3A-BE3 comprising eA3A linked to the N-terminus of nCas9 for comparison. Following overexpression of plasmids encoding CE_16_eA3A and eA3A-BE3 in Escherichia coli, we found that the Cas-embedded CBE showed increased expression compared to the N-terminally linked version (FIG. 5A). The purity of CE_16_eA3A or eA3A-BE3 protein was estimated to be ~97-99% determined using SDS-PAGE and gel staining (FIG. 5B). Highly purified CE_16_eA3A could routinely be produced at a yield of ~2 mg/L, which is approximately double what could be achieved with eA3A-BE3 (~1 mg/L) (FIG. 5C). The CE_16_eA3A protein could also be concentrated to ~40 mg/mL, which is double the maximal concentration that we could reach with eA3A-BE3 without visible aggregation or loss of editing activity.

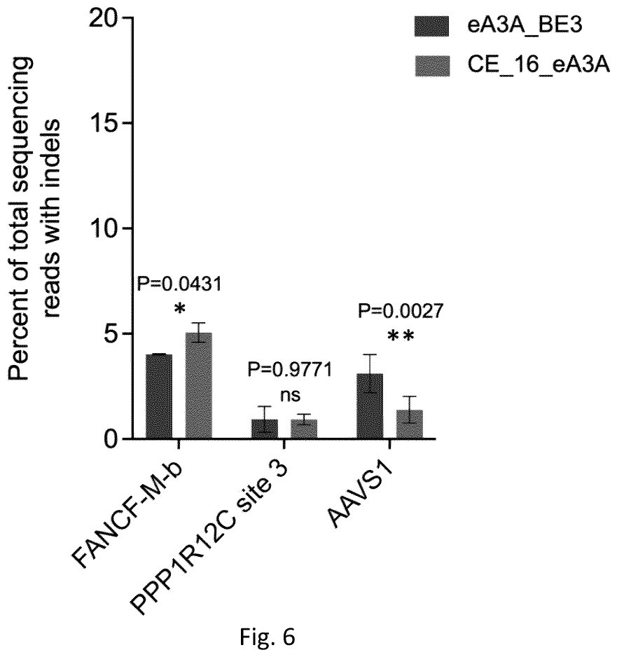

We first confirmed the activity of our purified CBEs using validated sgRNAs to target the FANCF-M-b site, PPP1R12C site 3, and AAVS1 site in HEK293T cells via RNP delivery (FIG. 1C). Three days after electroporation, we extracted genomic DNA and amplified the target genomic site for high-throughput sequencing. For all the tested sites, both CE_16_eA3A RNPs and eA3A-BE3 RNPs preferentially edited cytosines in the TC motif context (TC>CC>GC>AC) as expected based on the known substrate specificity of A3A (Silvas, Hou et al. 2018, Arbab, Shen et al. 2020) (Hou, Lee et al. 2021). High-throughput sequencing data showed that the C-to-T editing efficiencies of CE_16_eA3A RNPs (53.5±1.2% for FANCF-M-b TC₆, 63.4±9.9% for AAVS1 TC₈) were 10.8-17.0% higher (p<0.0001) than that of eA3A-BE3 RNPs (42.6±3.8% for FANCF-M-b TC₆, 46.4±2.3% for AAVS1 TC₈) for TC dinucleotides in the FANCF-M-b and AAVS1 sites, and comparable (p=0.7518) at the PPP1R12C site 3 TC₇. Despite higher activity, the indel level formed by CE_16_eA3A RNPs at the AAVS1 site (1.4±0.6%) was 2.2 fold-lower than that of eA3A-BE3 RNPs (3.1±0.9%). At FANCF-M-b and PPP1R12C site 3, indels caused by CE_16_eA3A RNPs (5.1±0.5% and 0.9±0.2) were similar with those of eA3A-BE3 (4.0±0.11% and 0.9±0.6) under these conditions (FIG. 6). These results indicate that CE_16_eA3A RNPs can effectively edit Cs in the TC motif context in human cells.

### Example 2: Editing Window of Cas9-Embedded Base Editors

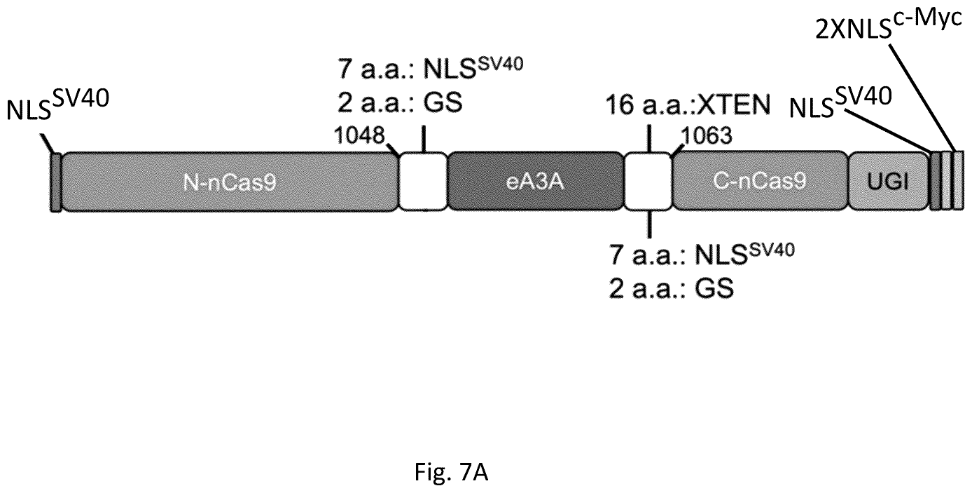

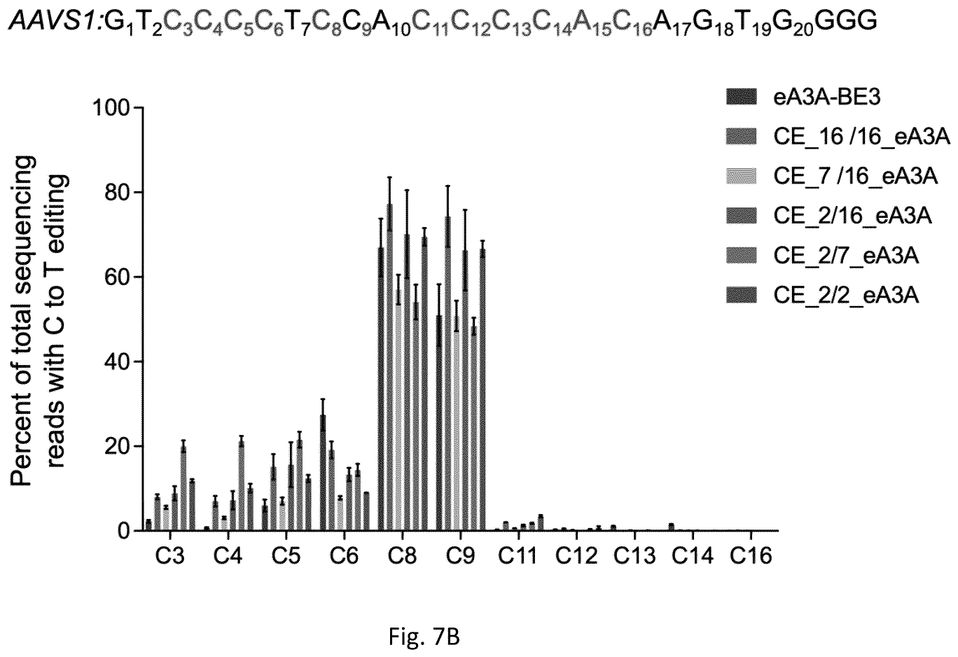

After establishing the activity of CE_16_eA3A RNPs, we next examined the effect of the linker length between Cas9 and eA3A on the editing efficiency and editing window of CE_CBEs. A structural model of CE-eA3A predicted by Alphafold (Jumper, Evans et al. 2021) predicted that eA3A inserted at the 1048Thr-1063Ile location of spCas9 could easily access the target ssDNA loop even with a short 2 amino acid (a.a.) Gly-Ser linker (“GS”), unlike N-terminally linked cytidine deaminases that require long flexible linkers (32 a.a.) to access a target ssDNA loop (Komor, Zhao et al. 2017). We therefore constructed and purified four additional CE base editor variants (CE_7_16_eA3A, CE_2_16_eA3A, CE_2_7_eA3A, and CE_2_2_eA3A) with N (amino) and C (carboxy) linker lengths varying between 16 and 2 a.a. and tested their C to T editing efficiencies as CE_CBE RNPs at the AAVS1 site. These variants were named CE_X_Y_eA3A (or CE_X/Y_eA3A, as in FIG. 7B), where X represents the amino acid length of a first linker flanking eA3A closest (compared to a second linker) to an amino-terminus of a CE base editor variant, and Y represents the amino acid length of the second linker, flanking eA3A, closest (compared to the first linker) to a carboxy-terminus of the CE base editor variant. Glycine-Serine (“GS”) was used as the 2 a.a. linker, SV40 NLS (SEQ ID NO:6) was used as the 7 a.a. linker, and XTEN (SEQ ID NO:5) was used as the 16 a.a. linker (FIG. 7A). Among all variants tested, CE_2_2_eA3A and CE_2_16_eA3A displayed similar C to T editing activity as the N-terminally linked eA3A-BE3 while the other variants had reduced C to T editing (14.8-19.2% at C8 and 0.4-5.1% at C9) compared to eA3A-BE3 (FIG. 7B (note that CE_16/16_eA3A is equivalent to the aforementioned CE_16_eA3A)). Therefore, we selected CE_2_2_eA3A with the shortest 2 a.a. N/C linker, henceforth named CE_2_eA3A, for further analysis.

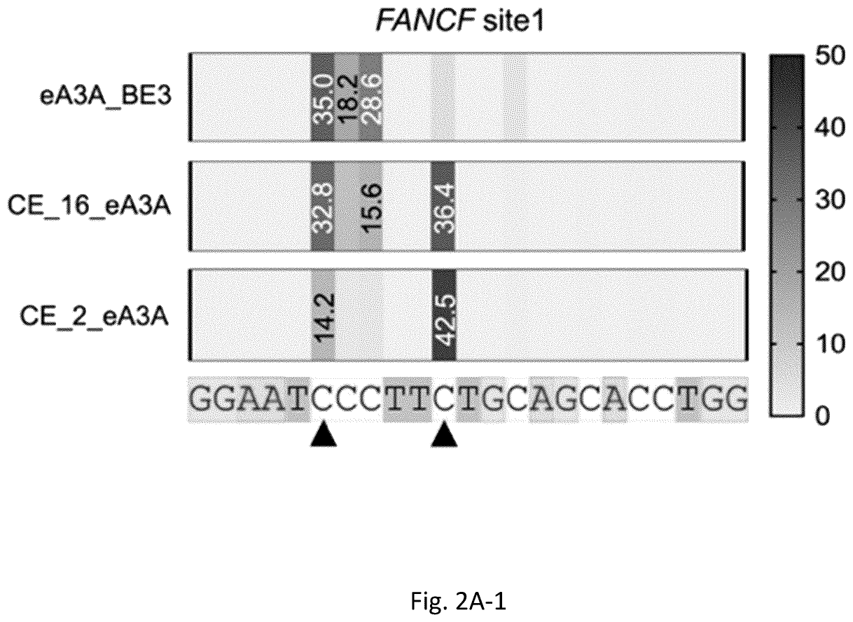

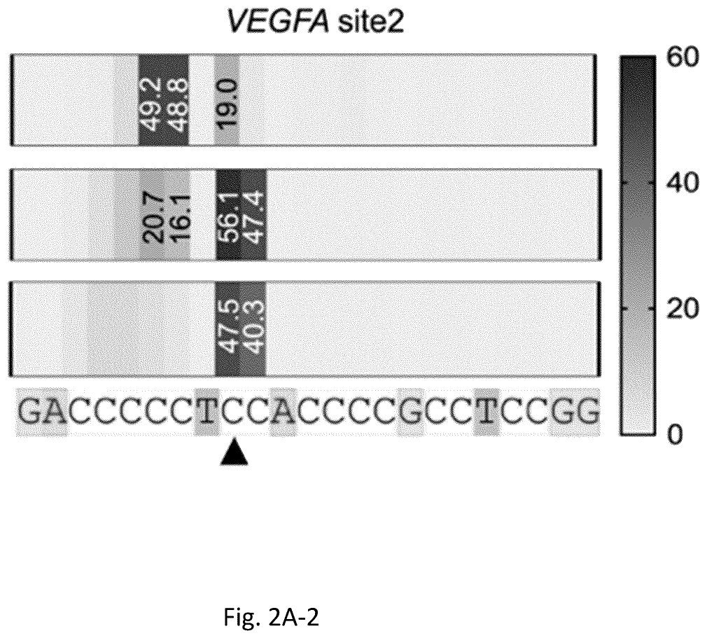

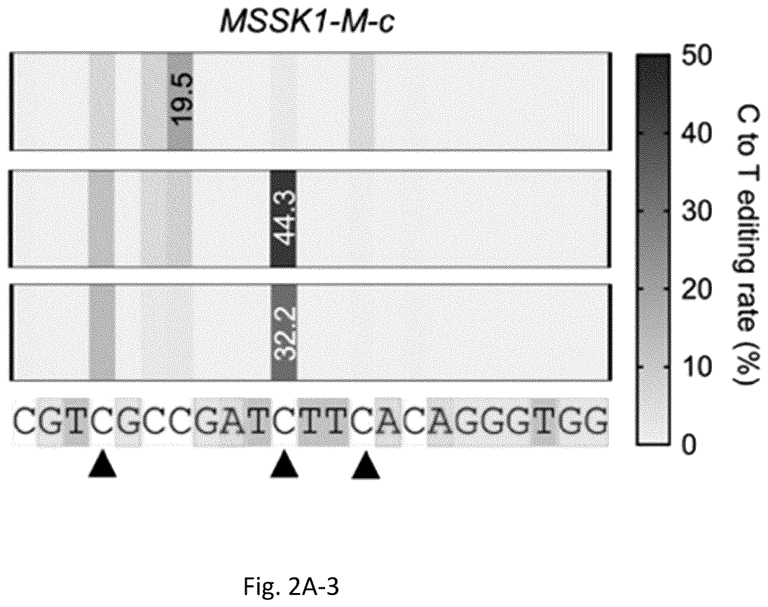

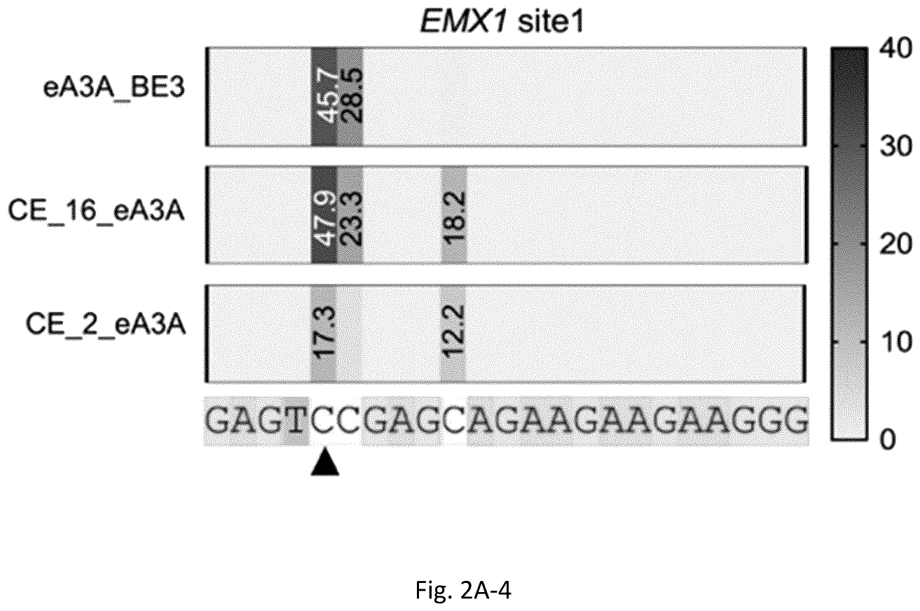

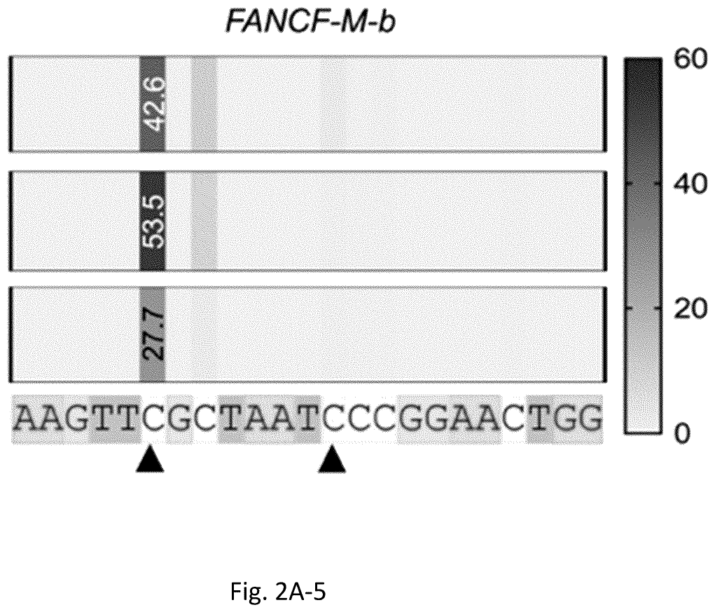

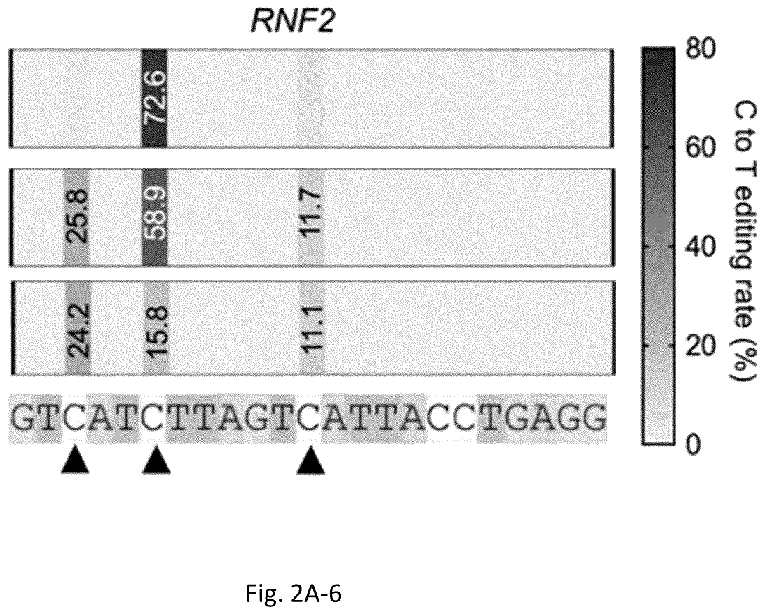

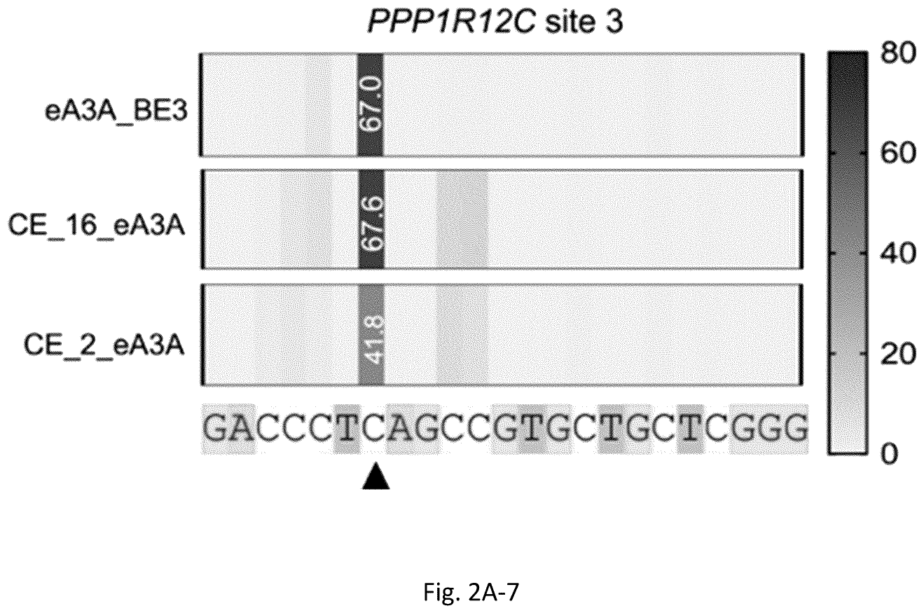

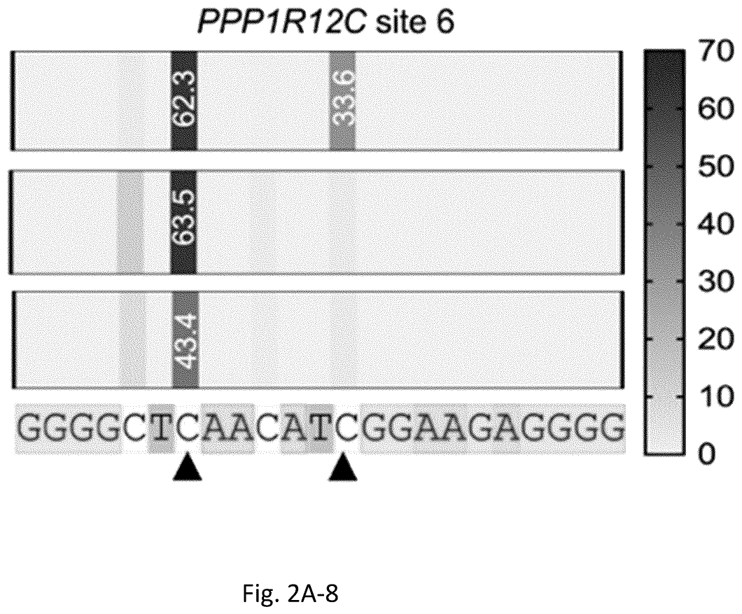

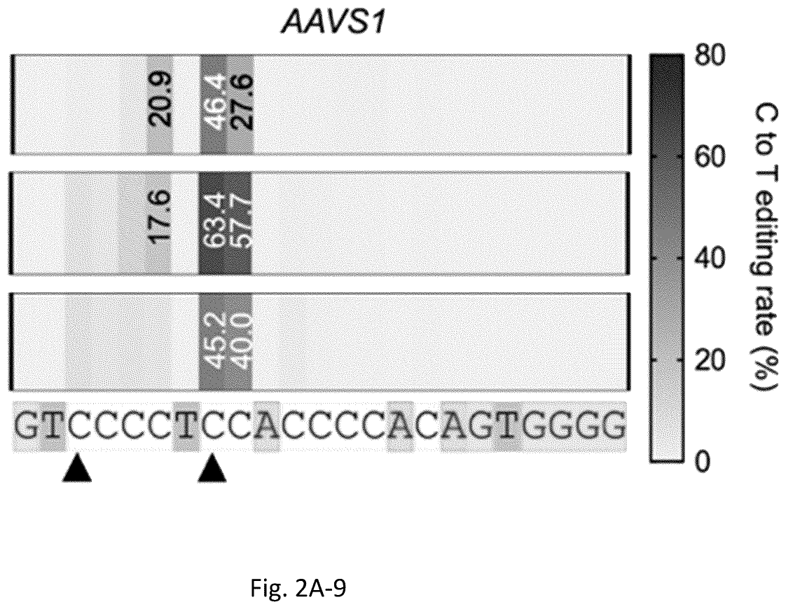

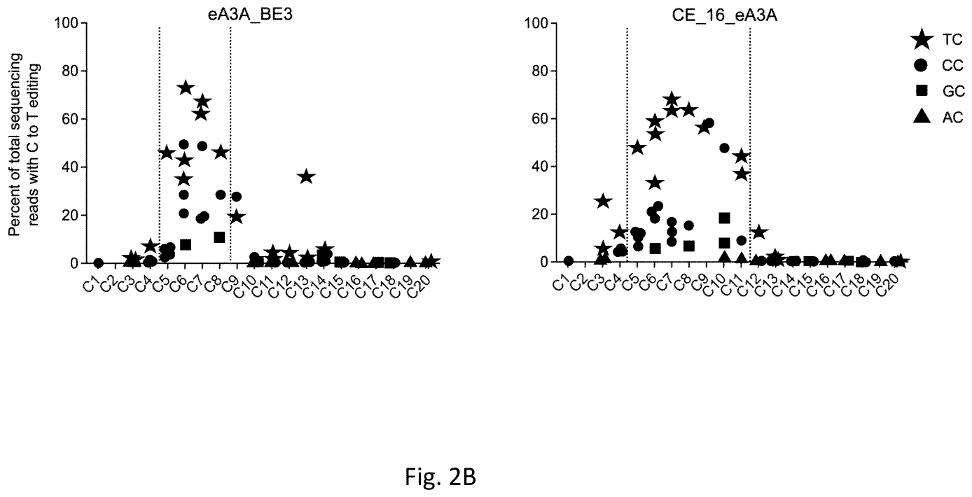

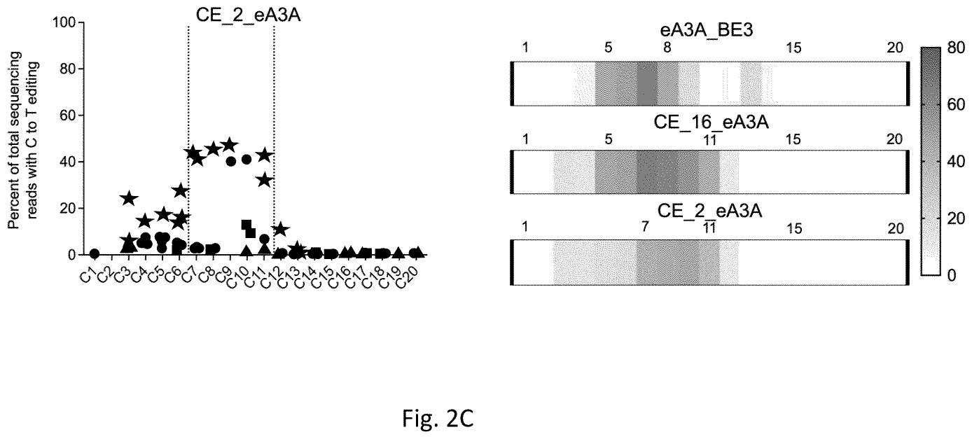

To further examine the editing activity windows of CE_16_eA3A and CE_2_eA3A RNPs in different nucleotide motif contexts, we analyzed average C-to-T base editing frequencies at 9 diverse target sites for our two CE_CBEs and eA3A_BE3 (Gehrke, Cervantes et al. 2018, Thuronyi, Koblan et al. 2019, Liu, Zhou et al. 2020) (FIG. 2). At FANCFsite1 with TC motifs at positions 5-6 and 10-11, eA3A-BE3 edited C6 (35.0±3.4%) more than C11 (4.7±0.3%), while CE_16_eA3A edited both positions to a similar extent (32.8±2.0% for C6, 36.1±1.4% for C11). By contrast, CE_2_eA3A had higher activity at C11 (42.5±1.8%) but low activity at C6 (14.2±2.7%). Similarly, in other target sites where the cytosine was present at position 5 or 6, CE_2_eA3A had significantly lower editing activity (e.g., 20.3±6.5% for EMXI TC5, FANCF-M-b TC6, and RNF2 TC6) compared to CE_16_eA3A and eA3A-BE3 (53.4±5.5% and 53.6±16.5%, respectively, at the same sites). At VEGFA site2 with TC motifs at positions 8-9 and MSSK1-M-c with TC motifs at positions 10-11, both CE_2_eA3A and CE_16_eA3A effectively edited cytosines (47.5±9.6% and 56.1±6.2% for VEGFA site2, 32.2±7.0% and 44.3±3.8% for MSSK1-M-c, respectively) while eA3A-BE3 was not efficient at these positions (19.0±4.5% for VEGFA site2, 1.5±0.1% for MSSK1-M-c). No base editor was active beyond protospacer position 13 (numbering from the 5′ end of the target site sequence), with the exception of eA3A-BE3 showing unexpected editing of TC13 at PPP1R12C site 6. Interestingly, the editing window (defined by half-maximal editing frequency) of CE_16_eA3A RNPs was expanded to protospacer positions 5 to 11 compared to that of eA3A-BE3 (positions ~5 to 8), while the editing window of CE_2_eA3A RNPs was shifted to positions 7 to 11. Thus, the editing activity window of CE base editors was shifted compared to the N-terminally fused eA3A-BE3, with CE_2_eA3A exhibiting a more distal editing window of similar size, while CE_16_eA3A had an expanded editing window that encompasses the areas edited by eA3A-BE3 and CE_2_A3A. Overall, these data indicate that CE_2_eA3A and CE_16_eA3A significantly expand the RNP-mediated CBE toolkit with shifted editing windows and high efficiency.

### Example 3: Dose-Dependent Editing and Target Product Purity

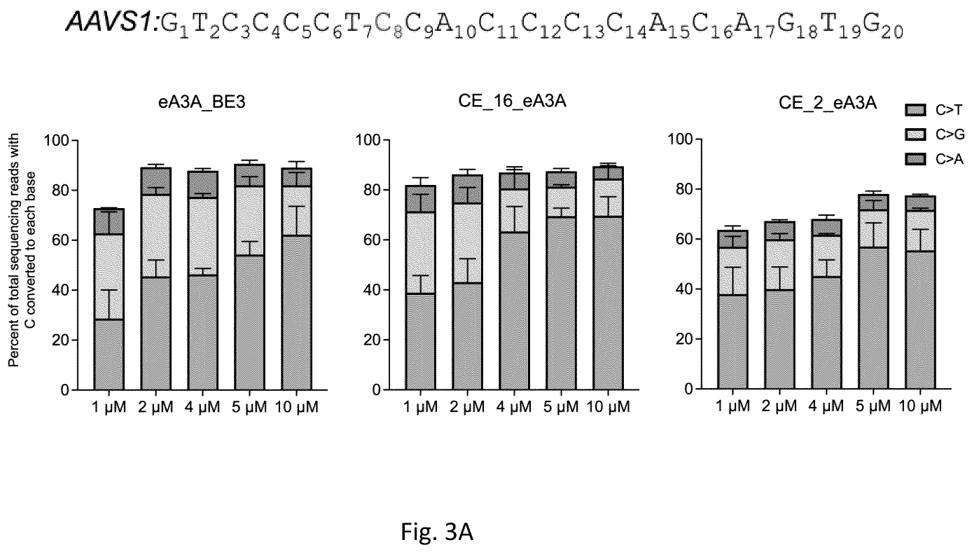

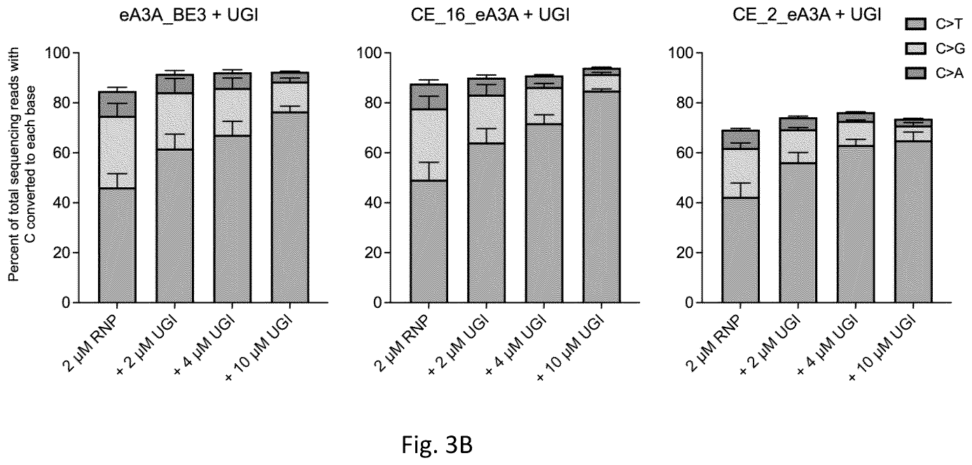

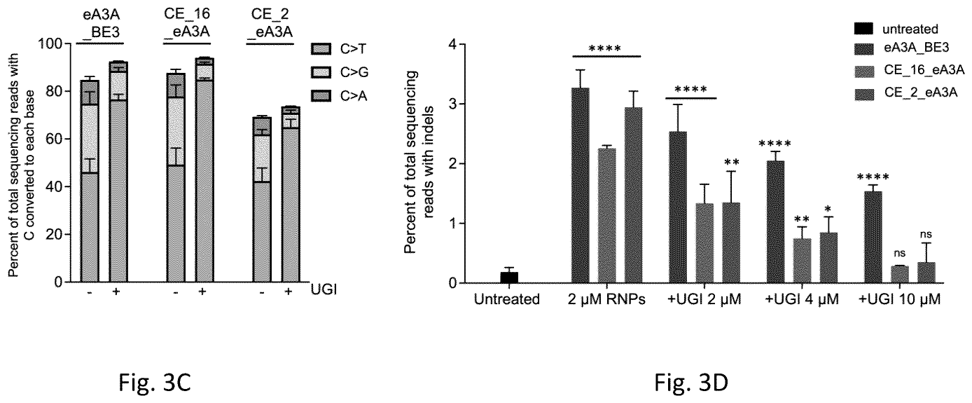

In contrast to plasmid delivery, RNP delivery of gene editing systems provides a major advantage by allowing for a more limited temporal window of editing activity, which has been shown to reduce cellular off-targets in other editing contexts (Cameron, Fuller et al. 2017). To examine this phenomenon with our CE_CBEs, we analyzed the editing frequencies after electroporation of AAVS1 sgRNA complexed with purified base editors at concentrations ranging from 1-10 μM (FIG. 3). Since all three effectors predominantly edited C8 at the AAVS1 site in our previous assay (FIG. 1B and FIG. 2A), we compared the editing frequencies at C8 for each CBE RNP across this concentration range in HEK293T cells. The total base editing yield (C to any other base) reached its maximum for all tested RNPs at around 2 μM (~90% for CE_16_eA3A and eA3A-BE3, 70% for CE_2_eA3A) and plateaued from 2-10 μM. However, the C-to-T base editing efficiency, or product purity, increased dramatically from 28.6-38.9% to 55.5-69.7% when the concentration of all tested RNPs was increased from 1 to 10 IM. At concentrations above 4 μM, CE_16_eA3A showed slightly higher C-to-T base editing efficiency compared to eA3A-BE3 and CE_2_eA3A. These results indicate that C-to-T base editing, or product purity—but not total base editing yield—had a strong correlation with concentration of the base editors. However, at 10 μM, all tested RNPs still produced some unwanted base substitutions (16.9±4.2% C-to-G and 6.0±1.7% C-to-A) and thus suboptimal product purity.

A recent study demonstrated that adding a plasmid encoding UGI to CBE RNPs increases C-to-T product purity by inhibiting uracil N-glycosylase activity in cells (Jang, Jo et al. 2021). However, it was not clear whether simply adding recombinant UGI protein to RNPs in trans can improve product purity and if so, how much UGI protein is required. To this end, we purified UGI protein containing a nuclear localization signal (NLS) at the C-terminus (UGI-NLS), and electroporated HEK293T cells with 2 μM CBE RNPs and UGI-NLS protein in molar ratios of 1:1, 2:1, or 5:1 (UGI:CBE). As expected, adding UGI-NLS protein to the RNPs did not further increase the total base editing yield but improved the editing purity in a dose-dependent manner (FIG. 3A and FIG. 3B). The addition of 5 molar equivalents of UGI-NLS protein to 2 μM CE_16_eA3A RNPs increased C to T editing from 43.1% to 85.0% (FIG. 3C), which is even higher than the C to T editing efficiency observed with 10 μM CE_16_eA3A RNP at the same site (70.0%). Similarly, the base editing product purity increased by adding UGI-NLS to RNPs for eA3A-BE3 and CE_2_eA3A. Interestingly, adding UGI-NLS protein to RNPs also decreased the formation of indels in a dose-dependent manner (FIG. 3D). Notably, indel formation was reduced to nearly background levels (0.19±0.08% for untreated cells) when UGI-NLS protein was added to 2 μM CE_eA3A RNPs in 5:1 ratio (0.28±0.15% for CE_16_eA3A, 0.35±0.32% for CE_2_eA3A; p>0.5 compared to untreated cells), in contrast to N-terminally fused base editor which still caused considerable indel formation (1.54±0.11%). These results suggest that the addition of UGI-NLS protein to Cas-embedded base editor RNPs at an optimal concentration can promote maximum C-to-T editing efficiency and purity while decreasing indel formation and unwanted base edits to background levels.

### Example 4: Off-Target Base Editing by RNPs in Mammalian Cells

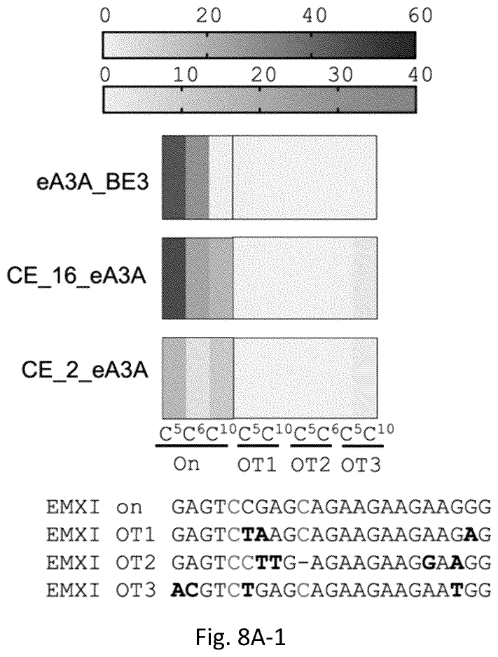

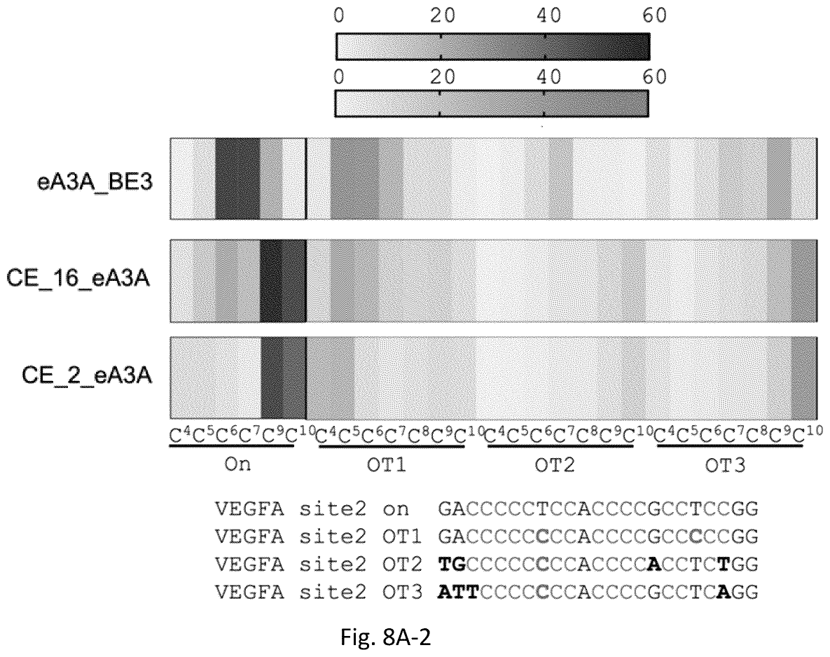

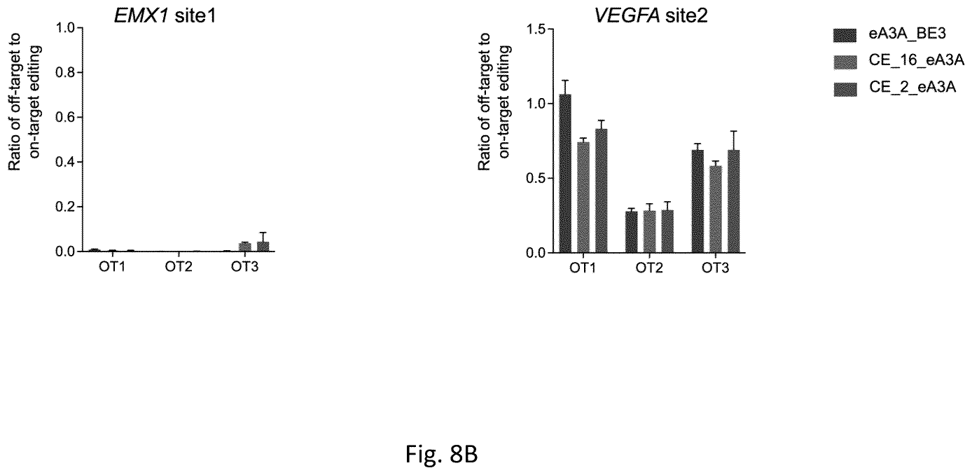

We reasoned that an expansion or shift in the editing window may not only have an effect on on-target base editing, but also the off-target editing profile of our new base editors. To investigate this, we performed targeted amplicon sequencing of six known off-target (OT) sites of two characterized sgRNAs (EMX1 or VEGFA site2) (FIG. 8A). At EMX1 OT1 and OT2, off targeting activities were undetectable while at OT3, the CE_eA3A RNPs showed slight off-targeting (2.0-3.0%) at C10 (found in a GC motif). Similarly, at VEGFA site2 OT1, OT2, and OT3, CE_eA3A RNPs exhibited slightly higher off-target levels than eA3A-BE3 RNPs at positions 9-10, but lower off-target levels at positions 5-7. At VEGFA site2 OT2 and OT3, cumulative off-target editing levels of CE_eA3A RNPs were similar to that of eA3A-BE3. However, CE_eA3A RNPs showed ~20-30% lower ratio of off-target to on-target editing than eA3A-BE3 at VEGFA site2 OT1 (FIG. 8B). Overall, the off-targeting positions by CE_16_eA3A and CE_2_eA3A were shifted compared to eA3A-BE3, suggesting that the editing window considerably affected off-target editing position. Thus, the altered editing window of Cas-embedded base editors can be leveraged to reduce off-target editing of concern especially at positions 5-7.

### Example 5: Base Editing of a Therapeutic Target in Human Hematopoietic Stem Cells

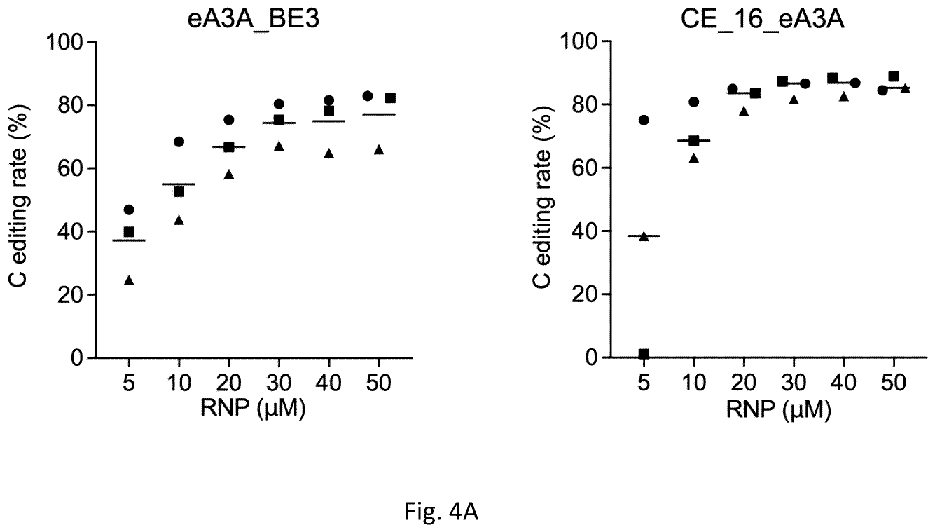

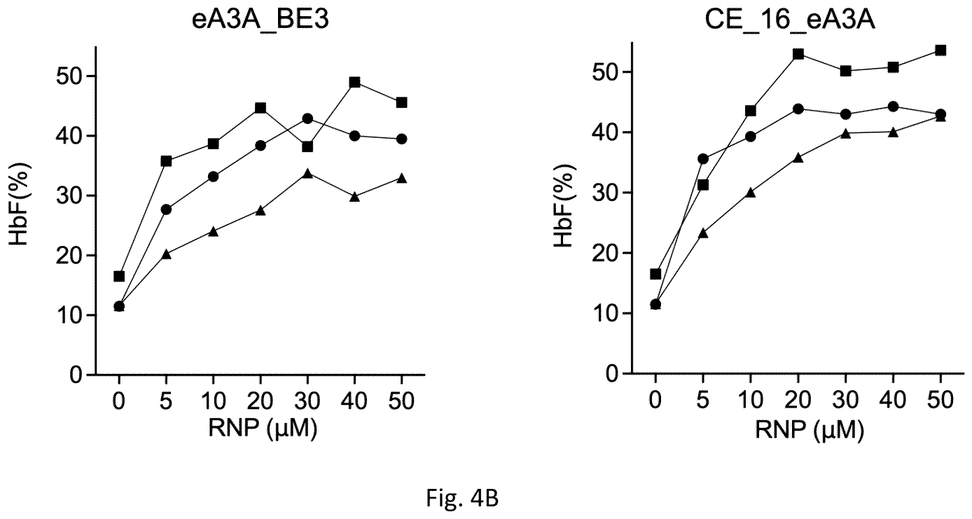

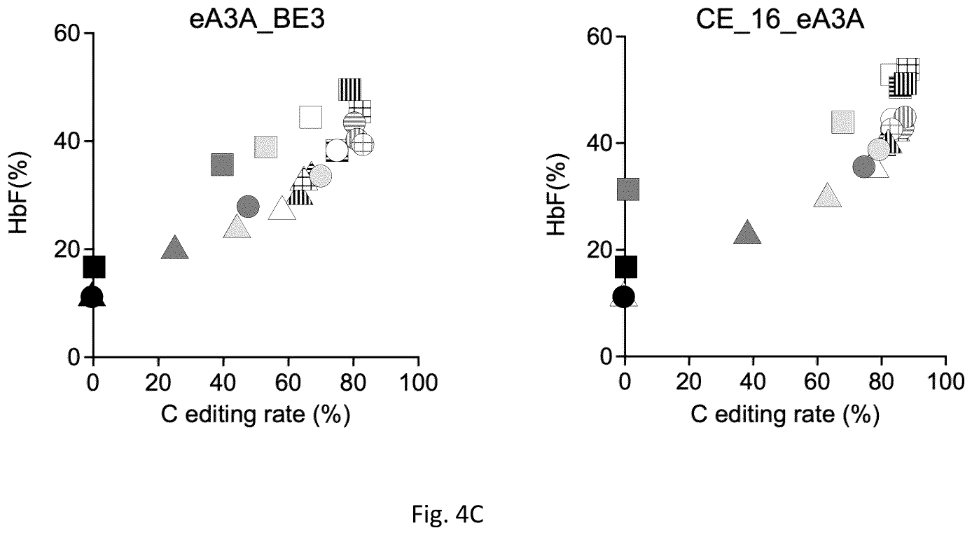

A previous study demonstrated the potential of A3A(N57Q)-CBE RNPs to edit a therapeutically relevant locus for sickle cell disease, however a high concentration of CBE RNP and two cycles of electroporation were required to achieve high editing rates (~90%) which reduced HSPC viability (~50%) and engraftment potential (Zeng, Wu et al. 2020). To examine whether our new CE-eA3A RNPs with improved solubility and activity could overcome these limitations, we targeted the +58 BCL11A erythroid enhancer region in human CD34+ HSPCs using a single electroporation (FIGS. 4A-C). Genetic disruption of the GATA1 motif within the enhancer sequence promotes therapeutic fetal hemoglobin (HbF) induction, which can ameliorate sickle cell disease and β-thalassemia (Rosanwo and Bauer 2021, White, Hart et al. 2022).

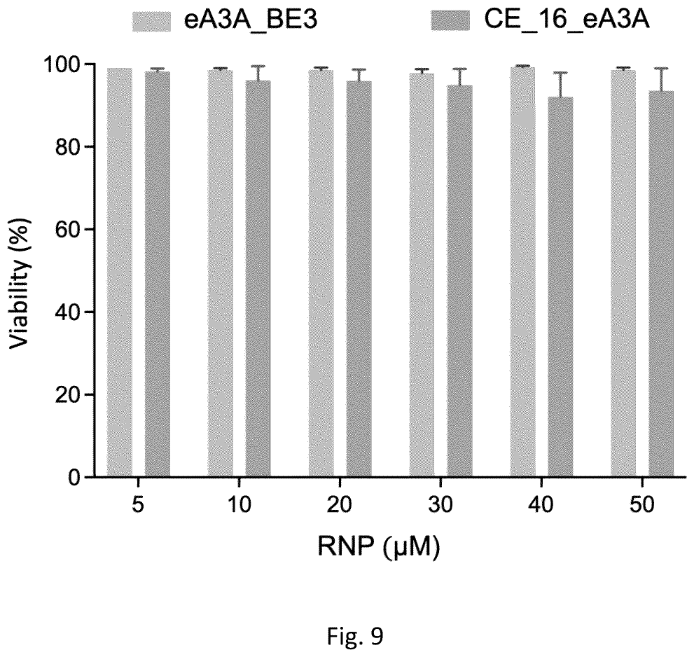

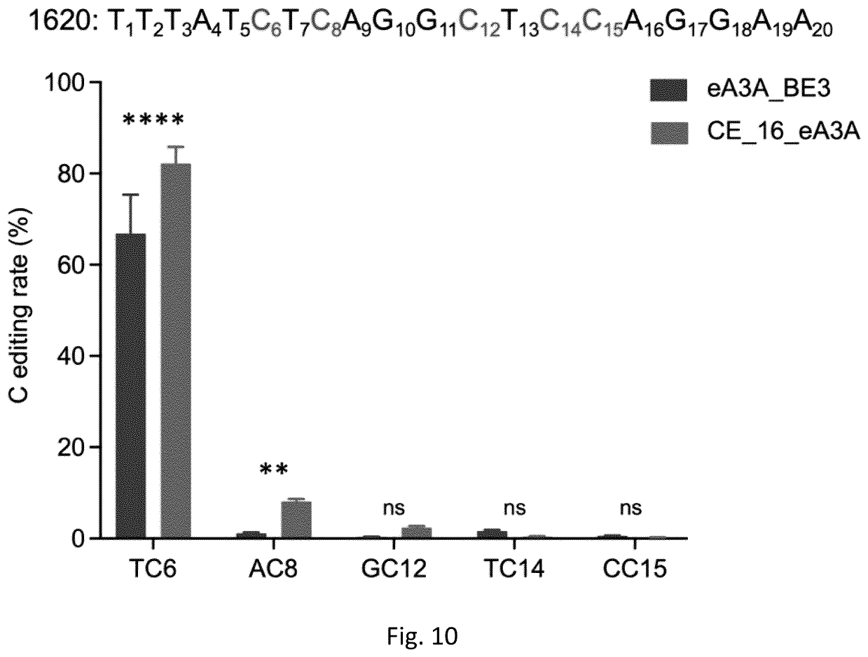

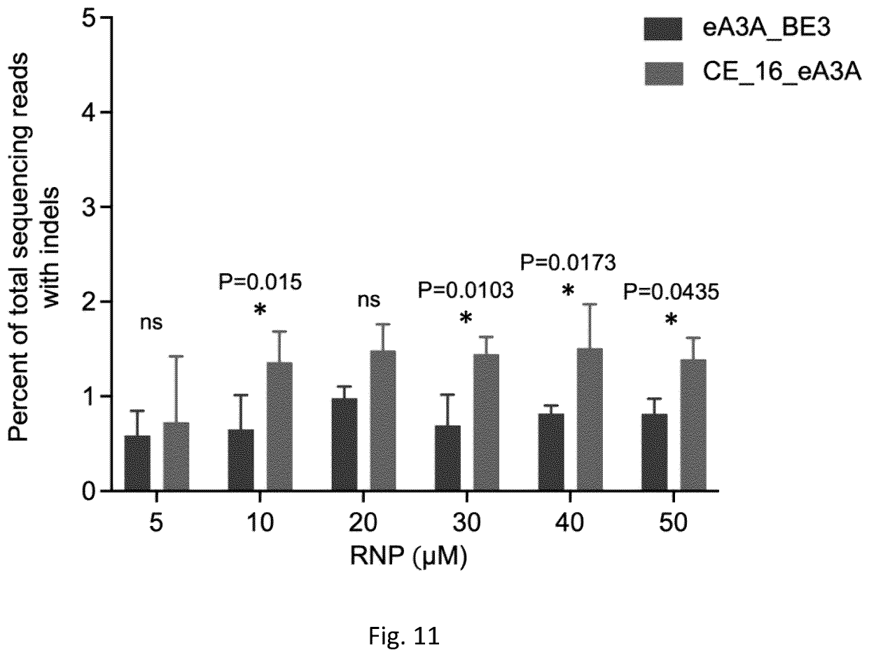

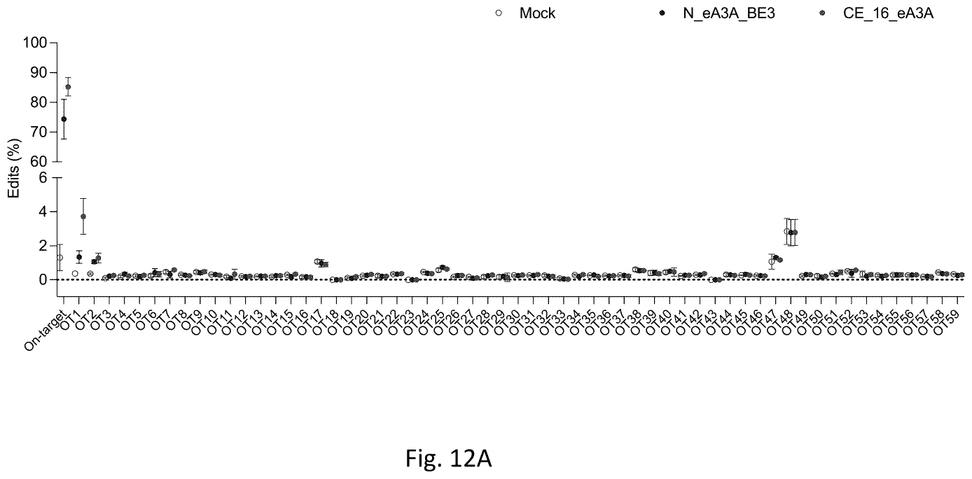

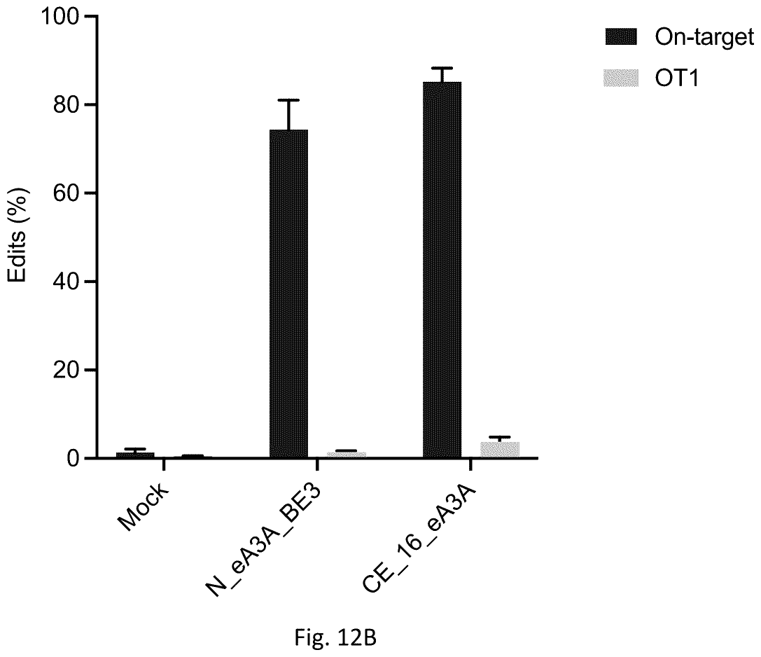

We electroporated human CD34+ HSPCs with CE_16_eA3A or eA3A-BE3 RNPs containing the previously validated sgRNA-1620 (Zeng, Wu et al. 2020) at concentrations varying from 5-50 μM. One day after electroporation, cell viability was ~91.5-99.5% for all tested concentrations of eA3A-BE3 and CE_16_eA3A, indicating that there was little detectable cellular toxicity (FIG. 9). As expected, both RNPs specifically edited the C6 position of the GATA binding motif with dose-dependent efficiency (FIG. 4A, FIG. 10). Notably, CE_16_eA3A RNPs achieved 9.1-16.3% higher editing rates compared to eA3A-BE3 RNPs at all tested doses. At a concentration of 20 μM, the CE_16_eA3A RNP produced 83.0±3.7% (C>T: 49.8±11.2%, C>G: 28.4±12.8%, C>A: 4.81±0.2%) base edits, which is similar to the editing efficiency of 50 μM eA3A-BE3 RNP (77.9±9.8%) and A3A(N57Q)-BE3 RNP previously reported (Zeng, Wu et al. 2020). At 30 μM, CE_16_eA3A RNPs yielded 86.0±3.1% base edits without substantial cellular toxicity (viability 95.0±3.9%) through a single electroporation, which was 8.1% higher than that of 50 μM eA3A-BE3 RNP. Despite improved editing activity, the indel levels formed by CE_16_eA3A RNPs at 5-50 μM were similar with that of eA3A-BE3 RNPs under the same conditions (1.0±0.1% for eA3A-BE3, 1.5±0.3% for CE_16_eA3A at 20 μM) (FIG. 11). Furthermore, CE_16_eA3A (or eA3A-BE3) RNPs did not induce any off-target editing at 58 of 59 potential off-target sites in HSPCs (Zeng, Wu et al. 2020) (FIGS. 12A and 12B). At only one site (OT1), low-level C editing was observed in cells edited with CE_16_eA3A (3.7±1.1%) or eA3A-BE3 (1.3±0.4%) compared to negative control (0.4±0.2%). The ratio of off-target to on-target C editing efficiencies by CE_16_eA3A (0.04) and eA3A-BE3 (0.02) was lower than that previously observed in cells edited by A3A(N57Q)-BE3 (Zeng, Wu et al. 2020).

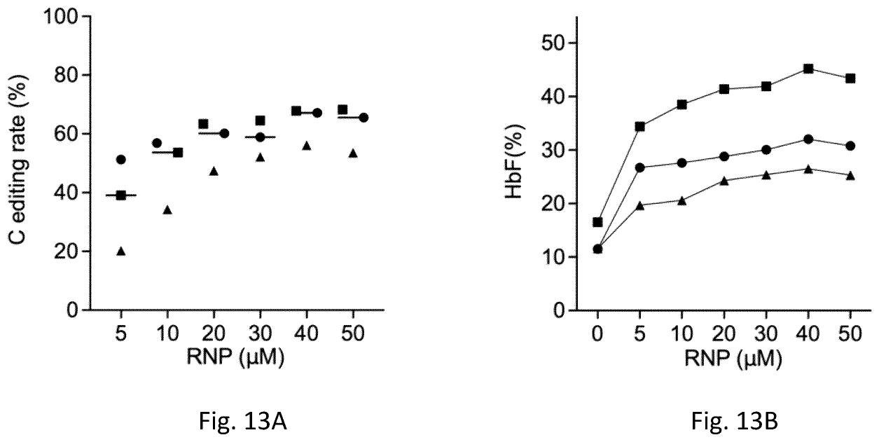

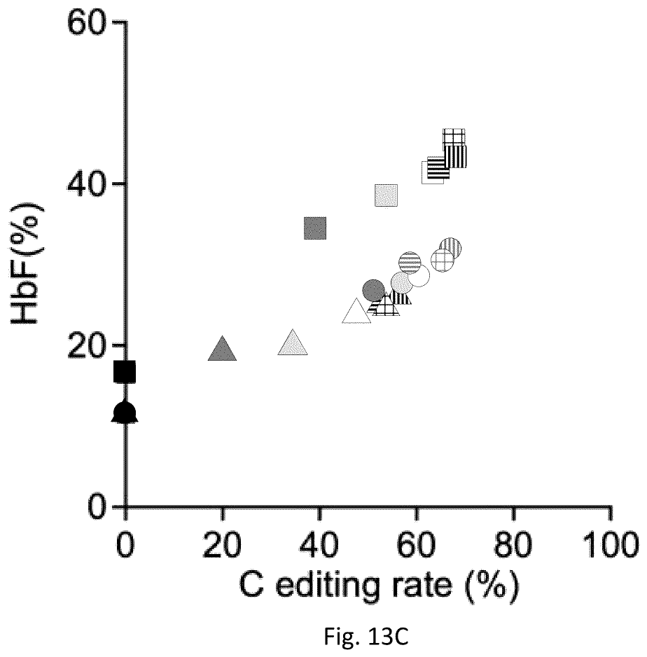

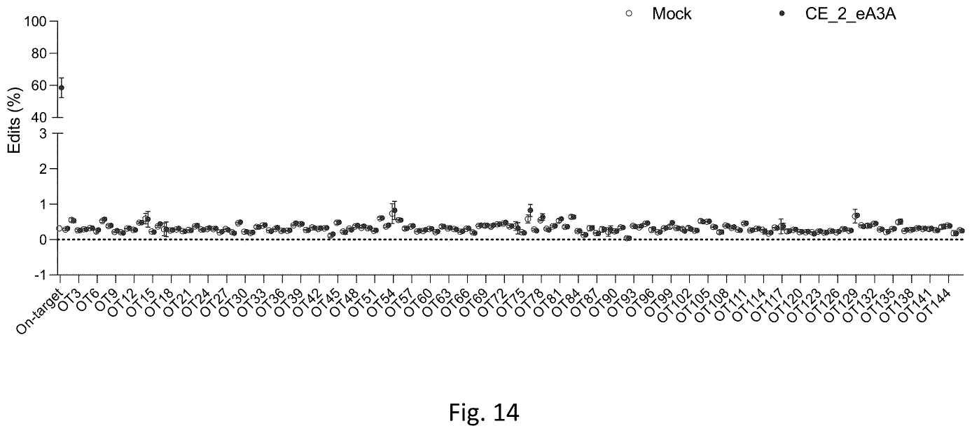

We also targeted the BCL11A enhancer region with CE_2_eA3A RNPs with a different sgRNA to test whether the shifted editing window can be leveraged to eliminate off-target editing. We used the previously characterized sgRNA-1618, which places the target cytosine of the GATA binding motif at position 11. CE_2_eA3A RNPs at 10 μM edited 48.7±12.3% C11, and at 30 μM produced 59.0±6.2% base edits, where A3A(N57Q)-BE3 RNP showed almost no activity under the same conditions with this sgRNA (Zeng, Wu et al. 2020) (FIGS. 13A-C). We investigated off-target editing by CE_2_eA3A at 146 potential genomic off-target sites for sgRNA-1618 identified by CasOFFinder tool (Bae, Park et al. 2014) within HSPCs edited with 30 μM CE_2_eA3A (FIG. 14). At all examined sites, there was no detectable editing by CE_2_eA3A RNPs relative to the negative control, indicating that CE_2_eA3A RNPs eliminated off-target editing while achieving efficient editing at the desired location.

Notably, the RNP-treated HSPCs expressed HbF when assessed on day 18 of erythroid culture (FIG. 4B), demonstrating a functional phenotype that correlated strongly with editing efficiency (FIG. 4C). At RNP concentrations above 30 μM, we were able to achieve 82.4-90.0% base editing by CE_16_eA3A that substantially elevated HbF protein levels by ~3.5-fold (35.9-54.6%) compared to the 13.2% (11.5-16.5%) baseline. Treatment with CE_16_eA3A resulted in up to 1.4-fold improvement in HbF induction compared to eA3A-BE3 and CE_2_eA3A RNPs at all tested doses.

### Example 6: Hematopoietic Stem Cell Engraftment in Mice

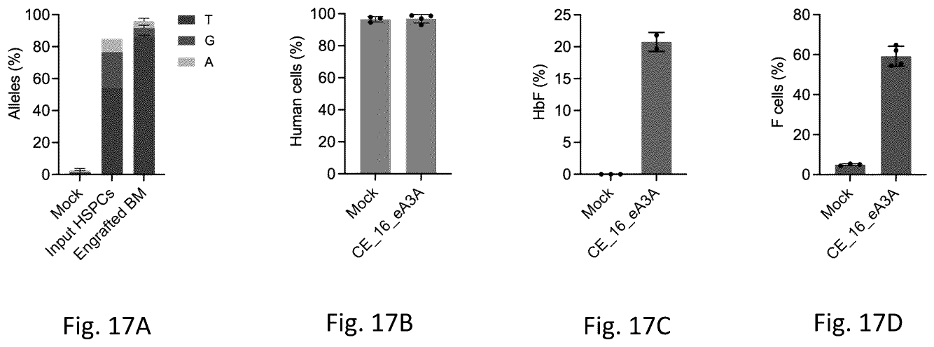

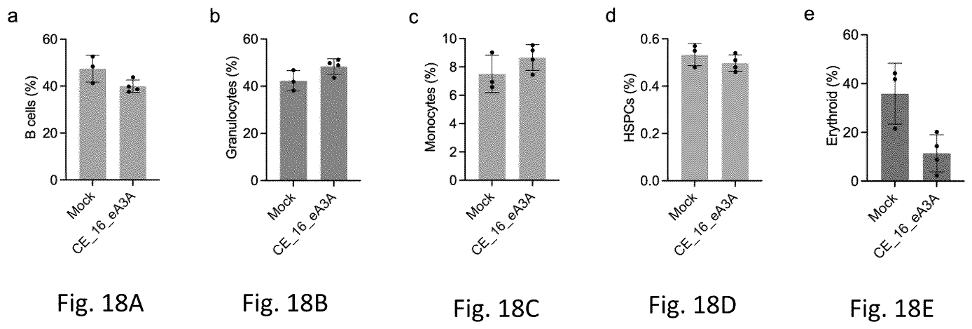

To further investigate the therapeutic potential of CE_16_eA3A editing, we infused human CD34+ HSPCs unedited or edited with CE_16_eA3A-sg1620 RNPs from one healthy donor into NBSGW mice. After 16 weeks, the editing efficiency and human hematopoietic engraftment were evaluated from isolated bone marrow (BM). The overall base editing frequency in engrafted BM was 96.0±0.8%, which is significantly higher than previously reported with two cycles of A3A(N57Q) RNP electroporation (Zeng, Wu et al. 2020) (FIG. 17A). We observed predominantly C to T (86.0±1.2%) base edits (5.5±1.9% to G and 4.5±1.7% to A). Flow cytometry analysis revealed ~96% human cells in BM from both unedited (96.5±1.7%) and edited mice (96.9±2.7%) (FIG. 17B), as well as similar relative abundances of human B cells, myeloid cells, T cells, and HSPCs in mice transplanted with unedited or edited HSPCs. These results indicate that base editing with CE_16_eA3A RNPs did not affect the ability of transplanted HSCs to successfully engraft or differentiate into multiple lineages. In engrafting erythroid cells from CE_16_eA3A-edited HSPCs, the HbF level was markedly increased from 0% to 20.8% and the proportion of fetal cells was substantially increased from 5.0% to 59.8% (FIGS. 17C and 17D). The transplantation of edited HSPCs into mice confirmed highly efficient editing in engrafting hematopoietic stem cells (HSCs), and, collectively, our findings indicate that CE_eA3A RNPs efficiently produce therapeutically relevant base edits in hematopoietic stem cells.

### Materials and Methods

### Plasmids and Oligonucleotides

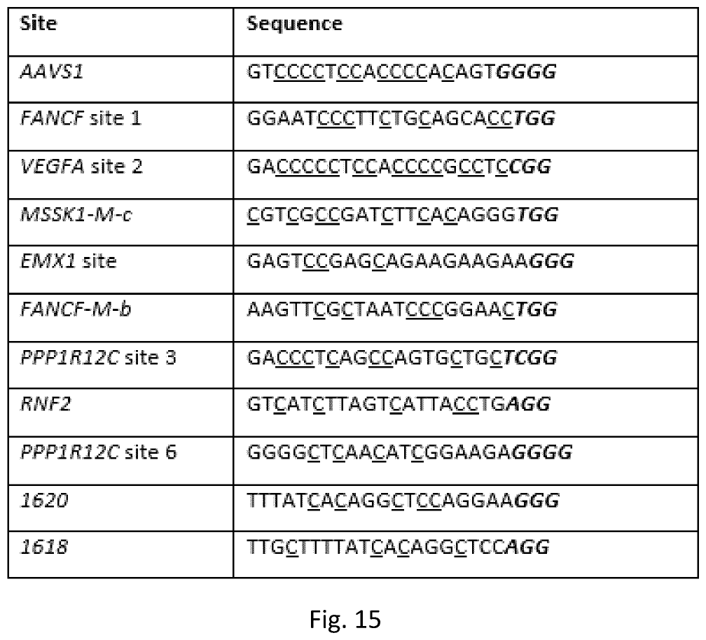

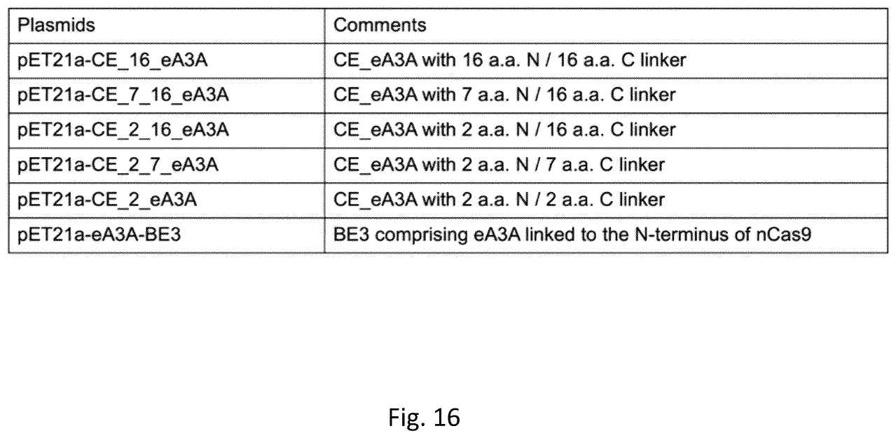

Expression plasmids used in this study are shown in FIG. 16. To construct pET21a-CE_2_eA3A, pET21a-CE_16_eA3A and pET21a-eA3A-BE3, we amplified eA3A, Cas9, and UGI genes from previously reported plasmids (addgene #131315) (Gehrke, Cervantes et al. 2018). We cloned the CBE constructs into the pET21a expression vector (Novagen) by standard molecular cloning methods. FIG. 15 shows target site sequences, including corresponding PAM sequences. Guide RNAs used for the experiments described herein are sgRNAs having the following structure: 5′-[guide sequence]-guuuuagagcuagaaauagcaaguuaaaauaaggcuaguccguuaucaacuugaaaaaguggcaccgagucggu gcuuuu-3′ (SEQ ID NO:15).

Expression and Purification of CE_2_eA3A, CE_16_eA3A and eA3A-BE3

For overexpression of CE_2_eA3A, CE_16_eA3A or eA3A-BE3 protein (Iyer, Suresh et al. 2019), the relevant plasmid was transformed into E. coli Rosetta2 (DE3) cells. The transformed cells were pre-cultured at 37° C. in LB medium with ampicillin (100 μg/mL) overnight, and the cell culture was inoculated into TB medium containing ampicillin (100 g/mL), grown at 37° C. When OD₆₀₀ value reached ~0.7-1.0, 0.5 mM IPTG was added to induce protein expression and the cell culture was further incubated overnight at 15° C. Cells were harvested by centrifugation at 5000 rpm for 20 min, resuspended in lysis buffer (50 mM Tris-HCl (pH 8.0), 1 μM NaCl, 10 mM imidazole, 5% glycerol, 1 mM TCEP, EDTA-free protease inhibitor pellet (1 capsule/50 ml, Roche)) and lysed by a cell disruptor. The lysate was clarified by ultracentrifugation at 18,000 rpm for 50 min. The supernatant was loaded onto a HisTrap FF column (GE Healthcare) equilibrated with lysis buffer, washed with washing buffer (50 mM Tris-HCl (pH 8.0), 1 μM NaCl, 20 mM imidazole, 5% glycerol, 1 mM TCEP) and followed by elution using elution buffer (50 mM Tris-HCl (pH 7.5), 500 mM NaCl, 500 mM imidazole, 1 mM TCEP) with a linear gradient of imidazole ranging from 0 mM to 500 mM. The eluted proteins were dialyzed overnight at 4° C. in 20 mM HEPES (pH 7.0), 150 mM NaCl, 10% glycerol, 1 mM TCEP. The dialyzed proteins were further purified using cation exchange column (5 ml, HiTrap-S, Buffer A=20 mM HEPES pH 7.0+1 mM TCEP, Buffer B=20 mM HEPES pH 7.0+1 μM NaCl+1 mM TCEP), and Superdex 200 size exclusion columns (running buffer A=20 mM HEPES pH 7.5, 150 mM NaCl, 10% glycerol or running buffer B=20 mM HEPES pH 7.5, 300 mM NaCl). The purified protein was concentrated using an Ultra-15 centrifugal filters ultracel-50K (Amicon) and flash frozen in liquid nitrogen.

### Cell Culture

Healthy human CD34 HSPCs were obtained from the Fred Hutchinson Cancer Research Center (Seattle, WA). Human CD34 HSPCs were thawed and cultured into serum-free medium Stem Cell Growth Medium (CellGenix, 20806-0500) supplemented with human Stem Cell Factor (SCF, 100 ng/ml) (CellGenix, 1418-050), FMS-like Tyrosine Kinase 3 Ligand (Flt3L, 100 ng/ml) (CellGenix, 1415-050) and Thrombopoietin (TPO, 100 ng/ml) (CellGenix, 1417-050). After 48 hours of pre-stimulation, HSPCs were harvested for electroporation. Electroporated cells were cultured in erythroid differentiation medium (EDM) consisting of IMDM (Gibco™, 12440061) supplemented with 330 μg/ml of Holo-Human Transferrin (Sigma-Aldrich, T0665-1G), 10 μg/ml of recombinant human insulin (Sigma-Aldrich, 19278-5ML), 2 IU/ml heparin (Sigma-Aldrich, H3149), 5% of human solvent detergent pooled plasma AB (Rhode Island Blood Center) and 3 IU/ml erythropoietin (AMGEM, 55513-144-10). During days 1-7 of culture, EDM was supplemented with 10-6M hydrocortisone (Sigma-Aldrich, H0135), 100 ng/ml human SCF (CellGenix, 1418-050) and 5 ng/ml of recombinant human IL-3 (PEPROTECH, 200-03). During days 7-11 of culture, EDM was supplemented with 100 ng/ml human SCF (CellGenix, 1418-050). During days 11-18 of culture, EDM had no additional supplements.

### RNP Electroporation

To examine RNP-mediated genome editing in HEK293T cells, CE_2_eA3A, CE_16_eA3A or eA3A-BE3 was complexed with a corresponding sgRNA, the corresponding sgRNA having a guide sequence identical to the target site sequences shown in FIG. 15, but lacking the last three nucleotides of the target site sequences (i.e., PAM sequences) shown in FIG. 15 and having thymine (T) replaced by uracil (U), in nuclease free water and incubated for 20 minutes at room temperature. Then, the resulting RNP complexes were mixed with HEK293T cells (1.0×10⁵) and electroporated using the neon transfection system.

For editing the +58 BCL11A erythroid enhancer region in human CD34+ HSPCs, electroporation was performed using Lonza 4D Nucleofector. The RNP complex was prepared by mixing 100 pmol-1000 pmol of N-eA3A, NC16:CE-eA3A or NC2:CE-eA3A with 300 pmol-3000 pmol of sgRNA-1620 (IDT, TTTATCACAGGCTCCAGGAA (SEQ ID NO: 16) guide sequence) or sgRNA-1618 (IDT, TTGCTTTTATCACAGGCTCC (SEQ ID NO: 17) guide sequence), adding 2% of glycerol and P3 solution up to 10 μl. The RNP was incubated at room temperature for 10-15 min. 50,000-100,000 cells were suspended in 10 μl of P3 solution. The cell suspensions were mixed with RNP and transferred to cuvette (Lonza 4D, V4XP-3032) for electroporation with program EO-100. The P3 solution was removed after 15 min of incubation at room temperature. The electroporated cells were cultured in EDM.

### Base Editing Measurements

To determine on/off-target editing frequencies in HEK293T cells, cells were harvested 3 days after electroporation and genomic DNA was extracted with 100 μL lysis buffer (10 mM Tris-HCl, pH 7.5, 0.05% SDS, 25 μg/mL proteinase K (NEB)) at 37° C. with incubation for 1 hour. Proteinase K was inactivated by 30-minute incubation at 80° C. The on- and off-target genomic sites (experimentally determined from a previous study) were PCR amplified with Phusion plus DNA polymerases (New England Biolabs) and locus-specific primers having tails complementary to the Truseq adapters: 98 degrees for 90 s; 30 cycles of 98 degrees for 15 s, 64 degrees for 30 s, and 72 degrees for 15 s; 72 degrees for 5 min. Resulting PCR products were subjected to illumina deep sequencing. For deep sequencing, resulting PCR products were amplified with index-containing primers to reconstitute the TruSeq adapters using Phusion plus DNA polymerases (98° C., 15 s; 62° C., 30 s; 72° C., 20 s)×10 cycles. E qual amounts of the PCR products from each experimental condition were pooled and gel purified. The purified library was deep sequenced using a paired-end 150 bp Illumina Mini Seq run. Frequencies of editing outcomes were quantified using CRISPResso2 (Clement, Rees et al. 2019). The percentage of editing was calculated as sequencing reads with the desired allele editing compared to all reads for the target locus.

To measure on-target editing frequencies of the +58 BCL11A erythroid enhancer region in human CD34⁺ HSPCs, cells were harvested 4-6 days after electroporation. Genomic DNA was isolated using the Blood and Tissue Kit (Qiagen, 69506) according to the vendor's recommendations. The BCL11A enhancer DHS +58 on-target region was amplified with KOD Hot Start DNA Polymerase (EMD-Millipore, 71086-31) and corresponding primers (forward primer 5′-AGAGAGCCTTCCGAAAGAGG-3′ (SEQ ID NO: 18) and reverse primer 5′ GCCAGAAAAGAGATATGGCATC-3′ (SEQ ID NO: 19)). The cycling conditions were 95° C. for 3 min; 30 cycles of 95° C. for 20 s, 60° C. for 10 s, and 70° C. for 10 s; 70° C. for 5 min. A total of 1 μl of locus specific PCR product was used for indexing PCR using KOD Hot Start DNA Polymerase (EMD-Millipore, 71086-31) and TruSeq i5 and i7 indexing primers (Illumina) following the cycling conditions: 95° C. for 3 min; 10 cycles of 95 CC for 20 s, 60° C. for 10 s, and 70° C. for 10 s; 70° C. for 5 min. The indexed PCR products were evaluated by Qubit dsDNA HS Assay Kit (Thermo Fisher, Q32854), TapeStation with High Sensitivity D1000 Reagents (Agilent, 5067-5585) and High Sensitivity D1000 ScreenTape (Agilent, 5067-5584) and KAPA Universal qPCR Master Mix (KAPA Biosystems, KK4824/Roche 07960140001). The products were pooled as equimolar and subjected to deep sequencing using MiniSeq (Illumina).

To quantify off-target editing frequencies within human HSPCs edited by CE_16_eA3A or CE_2_eA3A or eA3A-BE3 RNPs, we performed amplicon deep sequencing of potential genomic off target sites in genomic DNA samples extracted from cells edited with CE_16_eA3A or CE_2_eA3A or eA3A-BE3 RNPs and from negative control cells. For sgRNA-1620, 59 potential genomic off target sites were identified previously (Zeng, Wu et al. 2020). For sgRNA-1618, 146 potential off-target sites with three or fewer genomic mismatches and no bulges were identified using the CasOFFinder tool. Off-target sites were amplified with rhAmpSeq Library Mix 1 (IDT) and using rhAmpSeq forward and reverse assay primer pools. The cycling conditions were: 95° C. for 10 min; 14 cycles of 95° C. for 15 s and 61° C. for 8 min, and 99.5° C. for 15 min. Locus specific PCR product was diluted to 1:20 and 11 μL was used for the indexing PCR with the cycling conditions: 95° C. for 3 min; 24 cycles of 95° C. for 15 s, 60° C. for 30 s and 72° C. for 30 s; and 72° C. for 1 min. The resulting PCR products were evaluated by Qubit dsDNA HS Assay Kit (Thermo Fisher, Q32854), TapeStation with High Sensitivity D1000 Reagents (Agilent, 5067-5585) and High Sensitivity D1000 ScreenTape (Agilent, 5067-5584) and KAPA Universal qPCR Master Mix (KAPA Biosystems, KK4824/Roche 07960140001). The products were pooled as equimolar and subjected to deep sequencing using NovaSeq (Illumina).

### Hemoglobin HPLC

Erythroid cells were harvested 18 days after erythroid differentiation. Hemolysates were prepared by vertexing the cell pellet with Hemolysate reagent (Helena Laboratories, 5125). The hemolysates were mixed with D10 reagent (BioRad) and loaded on D10 Hemoglobin Testing System for the human hemoglobins.

### Human CD34+ HSPC Transplant and Engraftment Analysis

NBSGW mice were infused with human CD34+ HSPCs by retro-orbital injection. Bone marrow (BM) was harvested 16 weeks after transplantation. For flow cytometry analysis, the BM was blocked with Human TruStain FcX™ (Biolegend, #422302) and TruStain fcX™ (anti-mouse CD16/32, Biolegend, #101320) for 15 minutes at room temperature, followed by a 30-minute incubation on ice with Fixable Viability Dye (Thermo fisher, #65-0865-18), mCD45 Monoclonal Antibody (Thermo fisher, #61-0451-82), Mouse Anti-Human CD45 (BD Bioscience, #560367), anti-human CD235a (BioLegend, #349104), anti-human CD19 (BioLegend, #302212), anti-human CD33 (BioLegend, #366608), anti-human CD34 (BioLegend, #343504), and anti-human CD3 (BioLegend, #300420). Samples were acquired and recorded on a BD FACSAria II and data were analyzed with FlowJo™ software.

For MACS hCD235a isolation, BM was incubated with hCD235a microbeads (Miltenyi, #130-050-501) for 15 minutes on ice, followed by LS column hCD235a positive selection (Miltenyi, #130-042-401). 20% of the hCD235a+ cells were analyzed for HbF content by flow cytometry. The cells were stained with Hoechst 33342 for 20 minutes at 37° C., fixed with 0.05% glutaraldehyde (Sigma, #G6257) in PBS at room temperature for 10 minutes, then permeabilized with 0.1% Triton X-100 (Life, #HFH10). After permeabilization, cells were stained with mouse-antihuman CD235a (BioLegend, #349115) and HbF (Life, #MHFH014) in 0.1% BSA/PBS for 30 minutes on ice. Samples were acquired and recorded on a BD FACSAria II and data were analyzed with FlowJo™ software. 80% of the hCD235+ cells were also analyzed for hemoglobin content by HPLC. The cells were lysed with hemolysate reagent (Helena Laboratories cat #5125) and HPLC analysis was performed with D-10 Hemoglobin Analyzer (Bio-Rad).

### REFERENCES

Anzalone, A. V., L. W. Koblan and D. R. Liu (2020). “Genome editing with CRISPR-Cas nucleases, base editors, transposases and prime editors.” Nat Biotechnol 38(7): 824-844.
Arbab, M., M. W. Shen, B. Mok, C. Wilson, Z. Matuszek, C. A. Cassa and D. R. Liu (2020). “Determinants of Base Editing Outcomes from Target Library Analysis and Machine Learning.” Cell 182(2): 463-480 e430.
Bae, S., J. Park and J. S. Kim (2014). “Cas-OFFinder: a fast and versatile algorithm that searches for potential off-target sites of Cas9 RNA-guided endonucleases.” Bioinformatics 30(10): 1473-1475.
Berrios, K. N., N. H. Evitt, R. A. DeWeerd, D. Ren, M. Luo, A. Barka, T. Wang, C. R. Bartman, Y. Lan, A. M. Green, J. Shi and R. M. Kohli (2021). “Controllable genome editing with split-engineered base editors.” Nat Chem Biol 17(12): 1262-1270.
Cameron, P., C. K. Fuller, P. D. Donohoue, B. N. Jones, M. S. Thompson, M. M. Carter, S. Gradia, B. Vidal, E. Garner, E. M. Slorach, E. Lau, L. M. Banh, A. M. Lied, L. S. Edwards, A. H. Settle, D. Capurso, V. Llaca, S. Deschamps, M. Cigan, J. K. Young and A. P. May (2017). “Mapping the genomic landscape of CRISPR-Cas9 cleavage.” Nat Methods 14(6): 600-606.
Chu, S. H., M. Packer, H. Rees, D. Lam, Y. Yu, J. Marshall, L. I. Cheng, D. Lam, J. Olins, F. A. Ran, A. Liquori, B. Gantzer, J. Decker, D. Born, L. Barrera, A. Hartigan, N. Gaudelli, G. Ciaramella and I. M. Slaymaker (2021). “Rationally Designed Base Editors for Precise Editing of the Sickle Cell Disease Mutation.” CRISPR J 4(2): 169-177.
Clement, K., H. Rees, M. C. Canver, J. M. Gehrke, R. Farouni, J. Y. Hsu, M. A. Cole, D. R. Liu, J. K. Joung, D. E. Bauer and L. Pinello (2019). “CRISPResso2 provides accurate and rapid genome editing sequence analysis.” Nat Biotechnol 37(3): 224-226.
Doman, J. L., A. Raguram, G. A. Newby and D. R. Liu (2020). “Evaluation and minimization of Cas9-independent off-target DNA editing by cytosine base editors.” Nat Biotechnol 38(5): 620-628.
Gehrke, J. M., O. Cervantes, M. K. Clement, Y. Wu, J. Zeng, D. E. Bauer, L. Pinello and J. K. Joung (2018). “An APOBEC3A-Cas9 base editor with minimized bystander and off-target activities.” Nat Biotechnol 36(10): 977-982.
Grunewald, J., R. Zhou, S. P. Garcia, S. Iyer, C. A. Lareau, M. J. Aryee and J. K. Joung (2019). “Transcriptome-wide off-target RNA editing induced by CRISPR-guided DNA base editors.” Nature 569(7756): 433-437.
Hou, S., J. M. Lee, W. Myint, H. Matsuo, N. Kurt Yilmaz and C. A. Schiffer (2021). “Structural basis of substrate specificity in human cytidine deaminase family APOBEC3s.” J Biol Chem 297(2): 100909,
Huang, T. P., G. A. Newby and D. R. Liu (2021). “Precision genome editing using cytosine and adenine base editors in mammalian cells.” Nat Protoc 16(2): 1089-1128.
Huang, T. P., K. T. Zhao, S. M. Miller, N. M. Gaudelli, B. L. Oakes, C. Fellmann, D. F. Savage and D. R. Liu (2019). “Circularly permuted and PAM-modified Cas9 variants broaden the targeting scope of base editors.” Nat Biotechnol 37(6): 626-631.
Iyer, S., S. Suresh, D. Guo, K. Daman, J. C. J. Chen, P. Liu, M. Zieger, K. Luk, B. P. Roscoe, C. Mueller, O. D. King, C. P. Emerson, Jr. and S. A. Wolfe (2019). “Precise therapeutic gene correction by a simple nuclease-induced double-stranded break.” Nature 568(7753): 561-565.
Jang, H. K., D. H. Jo, S. N. Lee, C. S. Cho, Y. K. Jeong, Y. Jung, J. Yu, J. H. Kim, J. S. Woo and S. Bae (2021). “High-purity production and precise editing of DNA base editing ribonucleoproteins.” Sci Adv 7(35).
Jin, S., Y. Zong, Q. Gao, Z. Zhu, Y. Wang, P. Qin, C. Liang, D. Wang, J. L. Qiu, F. Zhang and C. Gao (2019). “Cytosine, but not adenine, base editors induce genome-wide off-target mutations in rice.” Science 364(6437): 292-295.
Jumper, J., R. Evans, A. Pritzel, T. Green, M. Figurnov, O. Ronneberger, K. Tunyasuvunakool, R. Bates, A. Zidek, A. Potapenko, A. Bridgland, C. Meyer, S. A. A. Kohl, A. J. Ballard, A. Cowie, B. Romera-Paredes, S. Nikolov, R. Jain, J. Adler, T. Back, S. Petersen, D. Reiman, E. Clancy, M. Zielinski, M. Steinegger, M. Pacholska, T. Berghammer, S. Bodenstein, D. Silver, O. Vinyals, A. W. Senior, K. Kavukcuoglu, P. Kohli and D. Hassabis (2021). “Highly accurate protein structure prediction with AlphaFold.” Nature 596(7873): 583-589.
Kim, S., D. Kim, S. W. Cho, J. Kim and J. S. Kim (2014). “Highly efficient RNA-guided genome editing in human cells via delivery of purified Cas9 ribonucleoproteins.” Genome Res 24(6): 1012-1019.
Koblan, L. W., J. L. Doman, C. Wilson, J. M. Levy, T. Tay, G. A. Newby, J. P. Maianti, A. Raguram and D. R. Liu (2018). “Improving cytidine and adenine base editors by expression optimization and ancestral reconstruction.” Nat Biotechnol 36(9): 843-846.
Komor, A. C., Y. B. Kim, M. S. Packer, J. A. Zuris and D. R. Liu (2016). “Programmable editing of a target base in genomic DNA without double-stranded DNA cleavage.” Nature 533(7603): 420-424.
Komor, A. C., K. T. Zhao, M. S. Packer, N. M. Gaudelli, A. L. Waterbury, L. W. Koblan, Y. B. Kim, A. H. Badran and D. R. Liu (2017). “Improved base excision repair inhibition and bacteriophage Mu Gam protein yields C:G-to-T:A base editors with higher efficiency and product purity.” Sci Adv 3(8): eaao4774.
Lee, S., N. Ding, Y. Sun, T. Yuan, J. Li, Q. Yuan, L. Liu, J. Yang, Q. Wang, A. B. Kolomeisky, I. B. Hilton, E. Zuo and X. Gao (2020). “Single C-to-T substitution using engineered APOBEC3G-nCas9 base editors with minimum genome- and transcriptome-wide off-target effects.” Sci Adv 6(29): eaba1773.
Liu, Y., C. Zhou, S. Huang, L. Dang, Y. Wei, J. He, Y. Zhou, S. Mao, W. Tao, Y.
Zhang, H. Yang, X. Huang and T. Chi (2020). “A Cas-embedding strategy for minimizing off-target effects of DNA base editors.” Nat Commun 11(1): 6073.
Long, J., N. Liu, W. Tang, L. Xie, F. Qin, L. Zhou, R. Tao, Y. Wang, Y. Hu, Y. Jiao, L. Li, L. Jiang, J. Qu, Q. Chen and S. Yao (2021). “A split cytosine deaminase architecture enables robust inducible base editing.” FASEB J 35(12): e22045.
Molla, K. A. and Y. Yang (2019). “CRISPR/Cas-Mediated Base Editing: Technical Considerations and Practical Applications.” Trends Biotechnol 37(10): 1121-1142.
Nishida, K., T. Arazoe, N. Yachie, S. Banno, M. Kakimoto, M. Tabata, M. Mochizuki, A. Miyabe, M. Araki, K. Y. Hara, Z. Shimatani and A. Kondo (2016). “Targeted nucleotide editing using hybrid prokaryotic and vertebrate adaptive immune systems.” Science 353(6305).
Oakes, B. L., D. C. Nadler, A. Flamholz, C. Fellmann, B. T. Staahl, J. A. Doudna and D. F. Savage (2016). “Profiling of engineering hotspots identifies an allosteric CRISPR-Cas9 switch.” Nat Biotechnol 34(6): 646-651.
Rees, H. A., A. C. Komor, W. H. Yeh, J. Caetano-Lopes, M. Warman, A. S. B. Edge and D. R. Liu (2017). “Improving the DNA specificity and applicability of base editing through protein engineering and protein delivery.” Nat Commun 8: 15790.
Rees, H. A. and D. R. Liu (2018). “Base editing: precision chemistry on the genome and transcriptome of living cells.” Nat Rev Genet 19(12): 770-788.
Rosanwo, T. O, and D. E. Bauer (2021). “Editing outside the body: Ex vivo gene-modification for beta-hemoglobinopathy cellular therapy.” Mol Ther 29(11): 3163-3178.
Silvas, T. V., S. Hou, W. Myint, E. Nalivaika, M. Somasundaran, B. A. Kelch, H. Matsuo, N. Kurt Yilmaz and C. A. Schiffer (2018). “Substrate sequence selectivity of APOBEC3A implicates intra-DNA interactions.” Sci Rep 8(1): 7511.
Suzuki, K., Y. Tsunekawa, R. Hernandez-Benitez, J. Wu, J. Zhu, E. J. Kim, F. Hatanaka, M. Yamamoto, T. Araoka, Z. Li, M. Kurita, T. Hishida, M. Li, E. Aizawa, S. Guo, S. Chen, A. Goebl, R. D. Soligalla, J. Qu, T. Jiang, X. Fu, M. Jafari, C. R. Esteban, W. T. Berggren, J. Lajara, E. Nunez-Delicado, P. Guillen, J. M. Campistol, F. Matsuzaki, G. H. Liu, P. Magistretti, K. Zhang, E. M. Callaway, K. Zhang and J. C. Belmonte (2016). “In vivo genome editing via CRISPR/Cas9 mediated homology-independent targeted integration.” Nature 540(7631): 144-149.
Tan, J., F. Zhang, D. Karcher and R. Bock (2019). “Engineering of high-precision base editors for site-specific single nucleotide replacement.” Nat Commun 10(1): 439. Thuronyi, B. W., L. W. Koblan, J. M. Levy, W. H. Yeh, C. Zheng, G. A. Newby, C. Wilson, M. Bhaumik, O. Shubina-Oleinik, J. R. Holt and D. R. Liu (2019). “Continuous evolution of base editors with expanded target compatibility and improved activity.” Nat Biotechnol 37(9): 1070-1079.
Wang, Y., R. Gao, J. Wu, Y. C. Xiong, J. Wei, S. Zhang, B. Yang, J. Chen and L. Yang (2019). “Comparison of cytosine base editors and development of the BEable-GPS database for targeting pathogenic SNVs.” Genome Biol 20(1): 218.
White, S. L., K. Hart and D. B. Kohn (2022). “Diverse Approaches to Gene Therapy of Sickle Cell Disease.” Annu Rev Med.
Wu, Y., J. Zeng, B. P. Roscoe, P. Liu, Q. Yao, C. R. Lazzarotto, K. Clement, M. A. Cole, K. Luk, C. Baricordi, A. H. Shen, C. Ren, E. B. Esrick, J. P. Manis, D. M. Dorfman, D. A. Williams, A. Biffi, C. Brugnara, L. Biasco, C. Brendel, L. Pinello, S. Q. Tsai, S. A. Wolfe and D. E. Bauer (2019). “Highly efficient therapeutic gene editing of human hematopoietic stem cells.” Nat Med 25(5): 776-783.
Yu, Y., T. C. Leete, D. A. Born, L. Young, L. A. Barrera, S. J. Lee, H. A. Rees, G. Ciaramella and N. M. Gaudelli (2020). “Cytosine base editors with minimized unguided DNA and RNA off-target events and high on-target activity.” Nat Commun 11(1): 2052.
Zafra, M. P., E. M. Schatoff, A. Katti, M. Foronda, M. Breinig, A. Y. Schweitzer, A. Simon, T. Han, S. Goswami, E. Montgomery, J. Thibado, E. R. Kastenhuber, F. J. Sanchez-Rivera, J. Shi, C. R. Vakoc, S. W. Lowe, D. F. Tschaharganeh and L. E. Dow (2018). “Optimized base editors enable efficient editing in cells, organoids and mice.” Nat Biotechnol 36(9): 888-893.
Zeng, J., Y. Wu, C. Ren, J. Bonanno, A. H. Shen, D. Shea, J. M. Gehrke, K. Clement, K. Luk, Q. Yao, R. Kim, S. A. Wolfe, J. P. Manis, L. Pinello, J. K. Joung and D. E. Bauer (2020). “Therapeutic base editing of human hematopoietic stem cells.” Nat Med 26(4): 535-541.
Zhou, C., Y. Sun, R. Yan, Y. Liu, E. Zuo, C. Gu, L. Han, Y. Wei, X. Hu, R. Zeng, Y. Li, H. Zhou, F. Guo and H. Yang (2019). “Off-target RNA mutation induced by DNA base editing and its elimination by mutagenesis.” Nature 571(7764): 275-278.
Zuo, E., Y. Sun, W. Wei, T. Yuan, W. Ying, H. Sun, L. Yuan, L. M. Steinmetz, Y. Li and H. Yang (2019). “Cytosine base editor generates substantial off-target single-nucleotide variants in mouse embryos.” Science 364(6437): 289-292.
Zuris, J. A., D. B. Thompson, Y. Shu, J. P. Guilinger, J. L. Bessen, J. H. Hu, M. L. Maeder, J. K. Joung, Z. Y. Chen and D. R. Liu (2015). “Cationic lipid-mediated delivery of proteins enables efficient protein-based genome editing in vitro and in vivo.” Nat Biotechnol 33(1): 73-80.

The publications (including patent publications), web sites, company names, books, manuals, treatise, and scientific literature referred to herein establish the knowledge that is available to those with skill in the art and are hereby incorporated by reference in their entirety to the same extent as if each was specifically and individually indicated to be incorporated by reference. Any conflict between any reference cited herein and the specific teachings of this specification shall be resolved in favor of the latter.

Various embodiments of the present invention may be characterized by the potential claims listed in the paragraphs following this paragraph (and before the actual claims provided at the end of this application). These potential claims form a part of the written description of this application. Accordingly, subject matter of the following potential claims may be presented as actual claims in later proceedings involving this application or any application claiming priority based on this application. Inclusion of such potential claims should not be construed to mean that the actual claims do not cover the subject matter of the potential claims. Thus, a decision to not present these potential claims in later proceedings should not be construed as a donation of the subject matter to the public.

Without limitation, potential subject matter that may be claimed (prefaced with the letter “P” so as to avoid confusion with the actual claims presented below) includes:
P1. A fusion protein comprising, in an N to C direction:
   (i) an N-terminal domain of a Cas9 nickase (N-nCas9);
   (ii) a first linker sequence;
   (iii) a cytidine deaminase domain comprising SEQ ID NO:2;
   (iv) a second linker sequence;
   (v) a C-terminal domain of the Cas9 nickase (C-nCas9); and
   (vi) a uracil glycosylase inhibitor sequence (UGI).
P2. The fusion protein of potential claim 1, wherein the N-terminal domain of the Cas9 nickase comprises an N-nCas9 amino acid sequence that is at least 85% identical to SEQ ID NO:1.
P3. The fusion protein of potential claim P2, wherein the N-terminal domain of the Cas9 nickase comprises SEQ ID NO:1.
P4. The fusion protein according to any one of potential claims P2 and P3, wherein the N-terminal domain of the Cas9 nickase consists of SEQ ID NO:1.
P5. The fusion protein according to any one of potential claims P1-P4, wherein the C-terminal domain of the Cas9 nickase comprises a C-nCas9 amino acid sequence that is at least 85% identical to SEQ ID NO:3.
P6. The fusion protein of potential claim P4, wherein the C-terminal domain of the Cas9 nickase comprises SEQ ID NO:3.
P7. The fusion protein according to any one of potential claims P5 and P6, wherein the C-terminal domain of the Cas9 nickase consists of SEQ ID NO:3.
P8. The fusion protein according to any one of potential claims P1-P7, wherein the uracil glycosylase inhibitor sequence comprises a UGI amino acid sequence that is at least 85% identical to SEQ ID NO:4.
P9. The fusion protein of potential claim P8, wherein the uracil glycosylase inhibitor sequence comprises SEQ ID NO:4.
P10. The fusion protein according to any one of potential claims P8 and P9, wherein the uracil glycosylase inhibitor sequence consists of SEQ ID NO:4.
P11. The fusion protein according to any one of potential claims P1-P10, wherein the first linker sequence consists of 2 to 16 amino acid residues.
P12. The fusion protein according to any one of potential claims P1-P11, wherein the first linker sequence is selected from the group consisting of Glycine-Serine, SEQ ID NO:5, and SEQ ID NO:6.
P13. The fusion protein according to any one of potential claims P1-P12, wherein the second linker sequence consists of 2 to 16 amino acid residues.
P14. The fusion protein according to any one of potential claims P1-P13, wherein the second linker sequence is selected from the group consisting of Glycine-Serine, SEQ ID NO:5, and SEQ ID NO:6.
P15. The fusion protein according to any one of potential claims P1-P14, wherein the fusion protein comprises a nuclear localization signal sequence (NLS).
P16, The fusion protein of potential claim 15, wherein the nuclear localization signal sequence is coupled to a terminus of the fusion protein selected from the group consisting of an N-terminus, a C-terminus, and combinations thereof
P17. The fusion protein according to any one of potential claims P15 and P16, wherein the nuclear localization signal sequence is selected from the group consisting of SEQ ID NO:6, SEQ ID NO:7, and combinations thereof.
P18. The fusion protein according to any one of potential claims P1-P17, wherein the cytidine deaminase domain consists of SEQ ID NO:2.
P19. The fusion protein according to any one of potential claims P1-P18, wherein the fusion protein comprises SEQ ID NO:8.
P20. The fusion protein according to any one of potential claims P1-P18, wherein the fusion protein comprises SEQ ID NO:9.
P21. The fusion protein according to any one of potential claims P1-P19, wherein the fusion protein consists of SEQ ID NO:8.
P22. The fusion protein according to any one of potential claims P1-P18 and P20, wherein the fusion protein consists of SEQ ID NO:9.
P23. A complex comprising a nucleic acid molecule and the fusion protein according to any one of potential claims P1-P22.
P24. The complex of potential claim P23, wherein the nucleic acid molecule is a single guide RNA.
P25. A method of treating a subject having or suspected of having a disease or disorder, the method comprising administering the fusion protein according to any one of potential claims P1-P22 ex vivo to a cell from the subject.
P26. The method of potential claim P25, wherein the N-terminal domain of the Cas9 nickase and the C-terminal domain of the Cas9 nickase of the fusion protein is bound to a single guide RNA.
P27. The method of potential claim P26, wherein the single guide RNA comprises a sequence of at least 10 contiguous nucleotides that is complementary to at least 10 contiguous nucleotides of a target sequence, the target sequence comprising a mutation associated with the disease or disorder.
P28. The method of potential claim P27, wherein the single guide RNA comprises a sequence of at least 18 contiguous nucleotides that is complementary to at least 18 contiguous nucleotides of a target sequence, the target sequence comprising a mutation associated with the disease or disorder.
P29. A method of treating a subject having or suspected of having a disease or disorder, the method comprising administering the complex according to any one of potential claims P23 and P24 ex vivo to a cell from the subject.
P30. The method of potential claim P29, wherein the nucleic acid molecule comprises a sequence of at least 10 contiguous nucleotides that is complementary to at least 10 contiguous nucleotides of a target sequence, the target sequence comprising a mutation associated with the disease or disorder.
P31. The method of potential claim P29, wherein the nucleic acid molecule comprises a sequence of at least 18 contiguous nucleotides that is complementary to at least 18 contiguous nucleotides of a target sequence, the target sequence comprising a mutation associated with the disease or disorder.
P32. The method according to any one of potential claims P25 to P31, the method further comprising co-administering a uracil glycosylase inhibitor protein (UGIP) ex vivo to the cell from the subject.
P33. The method of potential claim P32, wherein the uracil glycosylase inhibitor protein comprises a UGIP amino acid sequence that is at least 85% identical SEQ ID NO:4.
P34. The method of potential claim P33, wherein the uracil glycosylase inhibitor protein comprises SEQ ID NO:4.
P35. The method according to any one of potential claims P32-P34, wherein the uracil glycosylase inhibitor protein comprises a UGIP nuclear localization signal sequence (NLS).
P36. The method of potential claim P35, wherein the UGIP nuclear localization signal sequence is coupled to a terminus of the uracil glycosylase inhibitor protein selected from the group consisting of an N-terminus, a C-terminus, and combinations thereof
P37. The method according to any one of potential claims P35 and P36, wherein the UGIP nuclear localization signal sequence is selected from the group consisting of SEQ ID NO:6, SEQ ID NO:7, and combinations thereof.

The embodiments of the invention described above are intended to be merely exemplary; numerous variations and modifications will be apparent to those skilled in the art. All such variations and modifications are intended to be within the scope of the present invention as defined in any appended claims.

1. A fusion protein comprising, in an N to C direction:
(i) an N-terminal domain of a Cas9 nickase (N-nCas9);
(ii) a first linker sequence;
(iii) a cytidine deaminase domain comprising SEQ ID NO:2;
(iv) a second linker sequence;
(v) a C-terminal domain of the Cas9 nickase (C-nCas9); and
(vi) a uracil glycosylase inhibitor sequence (UGI).

2. The fusion protein of claim 1, wherein the N-terminal domain of the Cas9 nickase comprises an N-nCas9 amino acid sequence that is at least 85% identical to SEQ ID NO:1.

3. The fusion protein of claim 2, wherein the N-terminal domain of the Cas9 nickase comprises SEQ ID NO:1.

4. The fusion protein of claim 2, wherein the N-terminal domain of the Cas9 nickase consists of SEQ ID NO:1.

5. The fusion protein of claim 1, wherein the C-terminal domain of the Cas9 nickase comprises a C-nCas9 amino acid sequence that is at least 85% identical to SEQ ID NO:3.

6. The fusion protein of claim 4, wherein the C-terminal domain of the Cas9 nickase comprises SEQ ID NO:3.

7. The fusion protein of claim 1, wherein the C-terminal domain of the Cas9 nickase consists of SEQ ID NO:3.

8. The fusion protein of claim 1, wherein the uracil glycosylase inhibitor sequence comprises a UGI amino acid sequence that is at least 85% identical to SEQ ID NO:4.

9. The fusion protein of claim 8, wherein the uracil glycosylase inhibitor sequence comprises SEQ ID NO:4.

10. The fusion protein of claim 8, wherein the uracil glycosylase inhibitor sequence consists of SEQ ID NO:4.

11. The fusion protein of claim 1, wherein the first linker sequence consists of 2 to 16 amino acid residues.

12. The fusion protein of claim 1, wherein the first linker sequence is selected from the group consisting of Glycine-Serine, SEQ ID NO:5, and SEQ ID NO:6.

13. The fusion protein of claim 1, wherein the second linker sequence consists of 2 to 16 amino acid residues.

14. The fusion protein according of claim 1, wherein the second linker sequence is selected from the group consisting of Glycine-Serine, SEQ ID NO:5, and SEQ ID NO:6.

15. The fusion protein according of claim 1, wherein the fusion protein comprises a nuclear localization signal sequence (NLS).

16. The fusion protein of claim 15, wherein the nuclear localization signal sequence is coupled to a terminus of the fusion protein selected from the group consisting of an N-terminus, a C-terminus, and combinations thereof.

17. The fusion protein of claim 15, wherein the nuclear localization signal sequence is selected from the group consisting of SEQ ID NO:6, SEQ ID NO:7, and combinations thereof.

18. The fusion protein of claim 1, wherein the cytidine deaminase domain consists of SEQ ID NO:2.

19. The fusion protein of claim 1, wherein the fusion protein comprises SEQ ID NO:8.

20. The fusion protein of claim 1, wherein the fusion protein comprises SEQ ID NO:9.

21-37. (canceled)
